(19) United States
(12) Patent Application Publication
(10) Pub. No.: US 2025/0051357 A1
(43) Pub. Date: Feb. 13, 2025

(54) SOLID FORMS OF A GPR119 AGONIST AND PROCESSES FOR THE PREPARATION THEREOF

(71) Applicant: MANKIND PHARMA LTD., New Delhi (IN)

(72) Inventors: Sazid ALI, Gurugram (IN); Rakesh Ishwar PATIL, Gurugram (IN); Ramji Lal YADAV, Gurugram (IN); Rahul DEV, Gurugram (IN); Santosh Kumar RAI, Gurugram (IN); Anil KUMAR, Gurugram (IN)

(21) Appl. No.: 18/799,656

(22) Filed: Aug. 9, 2024

Related U.S. Application Data
(63) Continuation of application No. PCT/IB2024/057709, filed on Aug. 8, 2024.

(30) Foreign Application Priority Data
Aug. 11, 2023 (IN) 202311054016

(51) Int. Cl. C07D 513/04; A61K 31/506

(52) U.S. Cl. CPC C07D 513/04; A61K 31/506

(57) ABSTRACT

The present invention relates to solid forms of the GPR119 agonist of Formula I and processes for their preparation thereof. In particular, the present invention relates to crystalline forms of the compound of Formula I and their preparation.

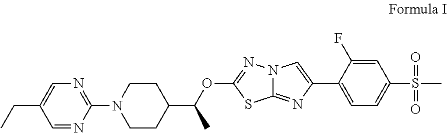 [SMILES: CCc1cnc(N2CCC([C@H](C)Oc3nn4cc(-c5ccc(S(C)(=O)=O)cc5F)nc4s3)CC2)nc1]

This application is a continuation of International Application No. PCT/IB2024/057709, filed Aug. 8, 2024, which claims the benefit of Indian Patent Application number 202311054016, filed on Aug. 11, 2023, each of which is hereby incorporated by reference.

### FIELD OF THE INVENTION

The present invention relates to solid forms of the GPR119 agonist of Formula I and processes for their preparation thereof. In particular, the present invention relates to crystalline forms of the compound of Formula I and their preparation.

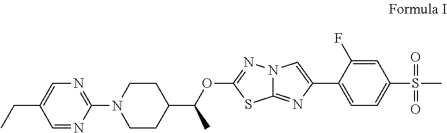 [SMILES: CCc1cnc(N2CCC([C@H](C)Oc3nn4cc(-c5ccc(S(C)(=O)=O)cc5F)nc4s3)CC2)nc1]

### BACKGROUND OF THE INVENTION

There are many drugs useful for the treatment of diabetes mellitus, including insulin, metformin, sulfonylureas, acarbose, thiazolidinedione, GLP-1 analogues and DPP IV inhibitors. Although a number of receptor classes exist in humans, by far the most abundant and therapeutically relevant is represented by the G protein-coupled receptor (GPCR) class. GPR119 is a cell-surface GPCR that is highly expressed in human (and rodent) islets as well as in insulin-secreting cell lines. Activation of GPR119 has been demonstrated to stimulate intracellular cAMP and lead to glucose dependent GLP-1 and insulin secretion (see, e.g., Soga et al., Biochem. Biophys. Res. Commun., 2005, 326).

U.S. Pat. No. 10,208,030 describes GPR119 agonists and processes for their preparation.

### SUMMARY OF THE INVENTION

In one aspect, the present invention relates to a solid form of a compound of Formula I:

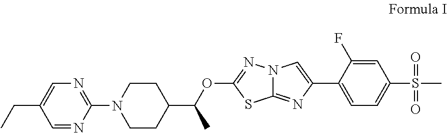 [SMILES: CCc1cnc(N2CCC([C@H](C)Oc3nn4cc(-c5ccc(S(C)(=O)=O)cc5F)nc4s3)CC2)nc1]

or a pharmaceutically acceptable salt thereof, wherein the solid form is selected from:
A. crystalline Form M-1 characterized by:
i) an X ray powder diffraction spectrum (XRPD) exhibiting characteristic peaks at 6.0±0.2, 8.1±0.2 and 17.3±0.2° 2θ;
ii) an XRPD substantially as depicted in FIG. 1;
iii) a differential scanning calorimetry (DSC) plot exhibiting endothermic peaks at about 111° C. and about 141.7° C.; or
iv) any combination of any of the foregoing;
B. crystalline Form M-2 characterized by:
i) an XRPD exhibiting characteristic peaks at 6.9±0.2, 10.6±0.2, 16.6±0.2, 19.2±0.2, 20.4±0.2, 25.8±0.2 and 26.4±0.2° 2θ;
ii) an XRPD substantially as depicted in FIG. 2;
iii) a DSC plot exhibiting an endothermic peak at about 144° C.; or iv) any combination of any of the foregoing;
C. crystalline Form M-3 characterized by:
i) an XRPD exhibiting characteristic peaks at 7.1±0.2, 13.3±0.2, 17.6±0.2 and 21.8±0.2° 2θ;
ii) an XRPD substantially as depicted in FIG. 3;
iii) a DSC plot exhibiting an endothermic peak at about 113° C.; or iv) any combination of any of the foregoing;
D. crystalline Form M-4 characterized by:
i) an XRPD exhibiting characteristic peaks at 8.8±0.2, 11.3±0.2, 17.8±0.2, 19.0±0.2, 23.1±0.2 and 25.8±0.2° 2θ;
ii) an XRPD exhibiting characteristic peaks at 4.4±0.2, 7.2±0.2, 8.8±0.2, 11.3±0.2, 13.3±0.2, 14.7±0.2, 15.6±0.2, 16.0±0.2, 17.8±0.2, 19.0±0.2, 20.4±0.2, 23.7±0.2 and 27.3±0.2° 2θ;
iii) an XRPD substantially as depicted in FIG. 4;
iv) a DSC plot substantially as depicted in FIG. 5;
v) a DSC plot exhibiting an endothermic peak at about 107° C.; or vi) any combination of any of the foregoing;
E. crystalline Form M-5 characterized by:
i) an XRPD exhibiting characteristic peaks at 4.3±0.2, 7.8±0.2, 10.2±0.2, 16.3±0.2 and 16.8±0.2° 2θ;
ii) an XRPD substantially as depicted in FIG. 6;
iii) a DSC plot exhibiting an endothermic peak at about 148° C.; or iv) any combination of any of the foregoing;
F. crystalline Form M-6 characterized by:
i) an XRPD exhibiting characteristic peaks at 5.6±0.2, 9.7±0.2 and 19.8±0.2° 2θ;
ii) an XRPD substantially as depicted in FIG. 7;
iii) a DSC plot exhibiting endothermic peaks at about 87° C., about 112° C. and about 142° C.; or
iv) any combination of any of the foregoing;
G. crystalline Form M-7 characterized by:
i) an XRPD exhibiting characteristic peaks at 4.3±0.2, 8.7±0.2, 10.6±0.2, 16.4±0.2 and 18.5±0.2° 2θ;
ii) an XRPD exhibiting characteristic peaks at 4.3±0.2, 8.7±0.2, 10.6±0.2, 16.4±0.2, 18.5±0.2, 19.5±0.2 and 21.9±0.2° 2θ;
iii) an XRPD substantially as depicted in FIG. 8;
iv) a DSC plot comprising endothermic peaks at about 100° C.; or v) any combination of any of the foregoing;
H. crystalline Form M-8 characterized by:
i) an XRPD exhibiting characteristic peaks at 4.6±0.2, 9.3±0.2, 12.3±0.2, 15.5±0.2 and 17.0±0.2° 2θ;
ii) an XRPD exhibiting characteristic peaks at 4.6±0.2, 6.4±0.2, 9.3±0.2, 12.3±0.2, 15.5±0.2, 17.0±0.2 and 17.7±0.2° 2θ;
iii) an XRPD substantially as depicted in FIG. 9;
iv) a DSC plot exhibiting an onset at about 127° C. and an endothermic peak at about 133° C.; or
v) any combination of any of the foregoing;
I. crystalline Form M-9 characterized by:
i) an XRPD exhibiting characteristic peaks at 11.5±0.2, 13.3±0.2, 17.9±0.2, 19.0±0.2, 23.2±0.2 and 25.8±0.2° 2θ;
ii) an XRPD exhibiting characteristic peaks at 7.2±0.2, 8.8±0.2, 11.5±0.2, 12.3±0.2, 13.3±0.2, 15.6±0.2, 16.0±0.2, 17.9±0.2, 19.0±0.2, 22.3±0.2, 23.2±0.2, 25.8±0.2, 27.3±0.2 and 29.4±0.2° 2θ;
iii) an XRPD substantially as depicted in FIG. 10;
iv) a DSC plot exhibiting endothermic peaks at about 108° C. and about 142° C.; or
v) any combination of any of the foregoing;
J. an amorphous form.

In one embodiment, the present invention relates to Form M-2 of the compound of Formula I, according to any of the embodiments described herein.

In one embodiment, the present invention relates to Form M-4 of the compound of Formula I, according to any of the embodiments described herein.

In one embodiment of any crystalline form or amorphous form of the compound of Formula I, the compound has a d₉₀ of from about 60 μm to about 100 μm.

In one embodiment of the solid forms described above:
a) Form M-1 further comprises one or more characteristic XRPD peaks at 4.5±0.2, 7.0±0.2, 10.5±0.2, 12.5±0.2, 14.9±0.2, 15.1±0.2, 15.4±0.2, 16.6±0.2, 18.6±0.2, 19.3±0.2, 20.4±0.2, 25.8±0.2 and 26.4±0.2° 2θ;
b) Form M-2 further comprises one or more characteristic XRPD peaks at 4.5±0.2, 12.5±0.2, 14.1±0.2, 14.9±0.2, 15.4±0.2, 17.9±0.2 and 20.8±0.2° 2θ;
c) Form M-3 further comprises one or more characteristic XRPD peaks at 4.3±0.2, 11.1±0.2, 15.2±0.2, 15.7±0.2, 16.0±0.2, 17.5±0.2, 18.8±0.2, 20.2±0.2, 21.7±0.2, 23.2±0.2 and 25.7±0.2° 2θ;
d) Form M-4 further comprises one or more characteristic XRPD peaks at 11.5±0.2, 12.3±0.2, 19.4±0.2, 22.0±0.2, 23.1±0.2, 24.5±0.2 and 25.9±0.2° 2θ;
e) Form M-5 further comprises one or more characteristic XRPD peaks at 7.6±0.2, 12.3±0.2, 17.8±0.2, 19.4±0.2 and 20.6±0.2° 2θ; and
f) Form M-6 further comprises one or more characteristic XRPD peaks at 15.9±0.2, 17.3±0.2, 18.6±0.2 and 20.3±0.2°.

In another aspect, the present invention relates to a process for the preparation of crystalline Form M-1 of the compound of Formula I or a pharmaceutically acceptable salt thereof according to any of the embodiments described herein, the process comprising:
a) dissolving a compound of Formula I in one or more solvents (e.g., one or more polar aprotic solvents, such as acetonitrile);
b) heating the reaction mixture (e.g., from a suitable temperature to reflux, such as heating to about 50° C. to about 70° C. for about 20 to about 30 minutes or until completely solubilized);
c) cooling the heated mixture obtained in step b) (e.g., to room temperature (about 25° C. to about 30° C.));
d) optionally seeding the reaction mixture with crystalline Form M-1 of the compound of Formula I; and
e) optionally isolating the crystalline Form M-1 of the compound of Formula I.

In another aspect, the present invention relates to a process for the preparation of crystalline Form M-2 of the compound of Formula I according to any of the embodiments described herein, the process comprising:
a) mixing racemic compound of Formula I in one or more solvents (e.g., one or more polar protic solvents, such as methanol);
b) stirring the reaction mixture (e.g., for a suitable time at a suitable temperature);
c) isolating the resulting solid (e.g., by filtering the reaction mixture);
d) obtaining the desired isomer of the crystalline Form M-2 of the compound of Formula I (e.g., performing a chiral resolution using chiral chromatography); and
e) optionally isolating the crystalline Form M-2 of the compound of Formula I.

In another aspect, the present invention relates to a process for the preparation of crystalline Form M-2 of the compound of Formula I according to any of the embodiments described herein, the process comprising heating a compound of Formula I (e.g., at a suitable temperature for a suitable time, such as at about 100-120° C. for about 2-4 hours) to give the crystalline Form M-2 of the compound of Formula I. In one embodiment, the process comprises heating amorphous or Form Ml of the compound of Formula I.

In another aspect, the present invention relates to a process for the preparation of crystalline Form M-3 of the compound of Formula I according to any of the embodiments described herein, the process comprising:
a) dissolving a compound of Formula I in one or more solvents (e.g., one or more polar protic solvents, such as ethanol);
b) heating the reaction mixture (e.g., heating to about 50° C. to about 70° C. for about 20 to about 30 minutes or until completely solubilized); and
c) optionally isolating the crystalline Form M-3 of the compound of Formula I.

In another aspect, the present invention relates to a process for the preparation of crystalline Form M-4 of the compound of Formula I according to any of the embodiments described herein, the comprising:
a) dissolving a compound of Formula I in a mixture of solvents (e.g., a combination of ethanol and heptane);
b) heating the reaction mixture (e.g., to a suitable temperature for a suitable time);
c) crystallizing the reaction mixture (e.g., by cooling to ambient temperature); and
d) optionally isolating the crystalline Form M-4 of the compound of Formula I.

In another aspect, the present invention relates to a process for the preparation of crystalline Form M-4 of the compound of Formula I according to any of the embodiments described herein, the process comprising:
a) mixing a compound of Formula I in one or more solvents (e.g., one or more polar aprotic solvents, such as acetonitrile);
b) stirring the reaction mixture (e.g., stirring at about 25° C. to about 30° C. for about 60 to about 120 minutes); and
c) optionally isolating the product of step b);
d) drying the product of step b) or c) (e.g., at about 50° C. to about 70° C. in vacuo);
e) stirring the dried product of step d) in a polar protic solvent (such as methanol);
f) drying the product of step e); and
g) optionally isolating the crystalline Form M-4 of the compound of Formula I.

In another aspect, the present invention relates to a process for the preparation of crystalline Form M-4 of the compound of Formula I according to any of the embodiments described herein, the process comprising heating crystalline Form M-3 of the compound of Formula I (e.g., at about 50° C. to about 70° C. for about 60 to about 70 hours) to give the crystalline Form M-4 of the compound of Formula I.

In another aspect, the present invention relates to a process for the preparation of crystalline Form M-5 of the compound of Formula I according to any of the embodiments described herein, the process comprising:
a) mixing a compound of Formula I in one or more solvents (e.g., a polar aprotic solvent, such as acetonitrile);
b) stirring the reaction mass at ambient temperature (e.g., for a suitable time); and
c) optionally isolating the crystalline Form M-5 of the compound of Formula I.

In another aspect, the present invention relates to a process for the preparation of crystalline Form M-6 of the compound of Formula I according to any of the embodiments described herein, the process comprising:
a) dissolving a compound of Formula I (e.g., Form M-2) in one or more solvents (such as xylene, heptane, nitromethane, or any combination thereof) under liquid nitrogen conditions (e.g., crash cooling to about −190° C.) to afford a solid mass;
b) maintaining the solid mass at room temperature to afford a clear solution (e.g., a solution with no visible particulates); and
c) optionally isolating the crystalline Form M-6 of the compound of Formula I.

In another aspect, the present invention relates to a process for the preparation of crystalline Form M-7 of the compound of Formula I according to any of the embodiments described herein, the process comprising:
a) dissolving a compound of Formula I in one or more solvents (e.g., one or more nonpolar aliphatic and/or aromatic hydrocarbons, such as a mixture of xylene and heptane);
b) heating the reaction mixture obtained in step a);
c) evaporating the solvent (e.g., maintaining at ambient temperature for about 24 to about 48 hours) to obtain crystalline Form M-7; and
d) optionally isolating the crystalline Form M-7 of the compound of Formula I.

In another aspect, the present invention relates to a process for the preparation of crystalline Form M-8 of the compound of Formula I according to any of the embodiments described herein, the process comprising:
a) mixing a compound of Formula I in a mixture of solvents (e.g., propanol and heptane);
b) heating the reaction mixture of step a);
c) cooling the reaction mixture obtained from step b); and
d) optionally isolating the crystalline Form M-8 of the compound of Formula I.

In another aspect, the present invention relates to a process for the preparation of crystalline Form M-9 of the compound of Formula I according to any of the embodiments described herein, the process comprising:
a) dissolving a compound of Formula I (such as, e.g., Form M-2) in one or more solvents (e.g., one or more polar protic solvents) under liquid nitrogen conditions (e.g., crash cooling to about −190° C.) to afford a solid mass;
b) maintaining the solid mass at room temperature to afford a clear solution (e.g., a solution with no visible particulates); and
c) optionally isolating the crystalline Form M-9 of the compound of Formula I.

In another aspect, the present invention relates to a process for the preparation of an amorphous form of the compound of Formula I according to any of the embodiments described herein, the process comprising:
a) providing a solution of a compound of Formula I in one or more solvents (e.g., acetone, acetonitrile, water, or any combination thereof);
b) lyophilizing the solution obtained in step a); and
c) optionally isolating the amorphous form of the compound of Formula I.

In another aspect, the present invention relates to a process for the preparation of an amorphous form of the compound of Formula I according to any of the embodiments described herein, the process comprising:
a) providing a solution of a compound of Formula I in one or more solvents;
b) removing the solvent (e.g., by evaporation or distillation); and
c) optionally isolating the amorphous form of the compound of Formula I.

In another aspect, the present invention relates to a process for the preparation of a premix of a compound of Formula I, the process comprising:
a) mixing one or more pharmaceutically acceptable excipients (e.g., hydroxypropyl methylcellulose), a compound of Formula I according to any of the embodiments described herein, and one or more solvents (e.g., dichloromethane);
b) removing the solvent (e.g., by evaporation); and
c) optionally isolating the premix of the compound of Formula I.

In certain embodiments, in any of the processes described herein, the compound of Formula I provided in step a) is selected from an amorphous form, a crystalline form, or any mixture thereof.

In another aspect, the present invention relates to a pharmaceutical composition comprising a solid form of a compound of Formula I according to any of the embodiments herein and a pharmaceutically acceptable excipient.

In another aspect, the present invention relates to a solid form of a compound of Formula I according to any of the embodiments herein, wherein the solid form is substantially free of other solid forms of the compound of Formula I described herein.

### BRIEF DESCRIPTION OF THE DRAWINGS

FIG. 1 shows the X-Ray Powder Diffraction (XRPD) pattern of the crystalline Form M-1 of the compound of Formula I of the present invention.

FIG. 2 shows the X-Ray Powder Diffraction (XRPD) pattern of the crystalline Form M-2 of the compound of Formula I of the present invention.

FIG. 3 shows the X-Ray Powder Diffraction (XRPD) pattern of the crystalline Form M-3 of the compound of Formula I of the present invention.

FIG. 4 shows the X-Ray Powder Diffraction (XRPD) pattern of the crystalline Form M-4 of the compound of Formula I of the present invention.

FIG. 5 shows the Differential Scanning calorimetry (DSC) of the crystalline Form M-4 of the compound of Formula I of the present invention.

FIG. 6 shows the X-Ray Powder Diffraction (XRPD) pattern of the crystalline Form M-5 of the compound of Formula I of the present invention.

FIG. 7 shows the X-Ray Powder Diffraction (XRPD) pattern of the crystalline Form M-6 of the compound of Formula I of the present invention.

FIG. 8 shows the X-Ray Powder Diffraction (XRPD) pattern of the crystalline Form M-7 of the compound of Formula I of the present invention.

FIG. 9 shows the X-Ray Powder Diffraction (XRPD) pattern of the crystalline Form M-8 of the compound of Formula I of the present invention.

FIG. 10 shows the X-Ray Powder Diffraction (XRPD) pattern of the crystalline Form M-9 of the compound of Formula I of the present invention.

FIG. 11 shows the X-Ray Powder Diffraction (XRPD) pattern of the stable amorphous form of the compound of Formula I of the present invention.

### DETAILED DESCRIPTION OF THE INVENTION

As used herein, the term “pharmaceutically acceptable salt” includes, but is not limited to, salts of inorganic acids, such as, e.g., hydrochloric acid, hydrobromic acid, sulphuric acid, phosphoric acid salt; salts of organic acids such as, e.g., succinic acid, formic acids, acetic acid, diphenyl acetic acid, triphenylacetic acid, caprylic acid, dichloroacetic acid, trifluoro acetic acid, propionic acid, butyric acid, lactic acid, citric acid, gluconic acid, mandelic acid, tartaric acid, malic acid, adipic acid, aspartic acid, fumaric acid, glutamic acid, maleic acid, malonic acid, benzoic acid, p-chlorobenzoic acid, nicotinic acid, o-hydroxybenzoic acid, p-hydroxybenzoic acid, 1-hydroxy-naphthalene-2-carboxylic acid, hydroxynaphthalene-2-carboxylic acid, ethanesulfonic acid, ethane-1,2-disulfonic acid, 2-hydroxyethane sulfonic acid, methanesulfonic acid, (+)-camphor-10-sulfonic acid, benzenesulfonic acid, naphthalene-2-sulfonic acid, p-toluenesulfonic acid; salts of pharmaceutically acceptable bases such as, e.g., metal salts including alkali metal or alkaline earth metal salts such as sodium, potassium, magnesium, calcium and zinc salts, ammonium salts; and any combination of any of the foregoing.

As used herein the term “premix” generally refers to a combination of a compound of Formula I according to any of the embodiments described herein and at least one pharmaceutically acceptable excipient (e.g., a pharmaceutically acceptable polymer, such as hydroxypropyl methylcellulose). In one embodiment, individual particles of the components cannot be distinguished in the premix, e.g., by using techniques such as optical microscopy.

As used herein, the term “excipient” or “pharmaceutically acceptable excipient” means a component of a pharmaceutical product that is not an active ingredient, and includes, but is not limited to fillers, diluents, disintegrants, glidants, stabilizers, surface active agents, polymers, carriers, etc. Excipients that are useful in preparing a premix or pharmaceutical composition as described herein are generally safe, non-toxic and neither biologically nor otherwise undesirable, and are acceptable for veterinary use as well as human pharmaceutical use. One excipient can perform more than one function.

In an embodiment, at least one pharmaceutically acceptable excipient is selected from the group consisting of polyvinyl pyrrolidone, povidone K-30, povidone K-60, Povidone K-90, methylcellulose, methacrylic acid copolymer, hydroxypropylmethyl cellulose, hydroxypropylmethyl cellulose phthalate, hydroxypropylmethyl cellulose acetate succinate (HPMC-AS), hydroxypropylmethyl cellulose, hydroxypropyl cellulose SSL (HPC-SSL), hydroxypropyl cellulose SL (HPC-SL), hydroxypropyl cellulose L (HPC-L), hydroxyethyl cellulose, Soluplus® (polyvinyl caprolactam-polyvinyl acetate-polyethylene glycol graft copolymer (PCL-PVAc-PEG)), gelucire 44/14, ethyl cellulose, D-alpha-tocopheryl polyethylene glycol 1000 succinate, cellulose acetate phthalate, carboxy methyl ethyl cellulose, sodium starch glycolate any combination of any of the foregoing.

As used herein, the term “substantially free” means a compound of Formula I (or a pharmaceutically acceptable salt thereof) wherein each impurity is present less than about 0.2% by area percentage of HPLC, such as less than about 0.15% by area percentage of HPLC. In one embodiment, the compound of Formula I (or a pharmaceutically acceptable salt thereof) is free of impurities (e.g., a particular impurity is not present in detectable amount by area percentage of HPLC).

As used herein, in one embodiment, the term “substantially pure” refers to the chemical purity of a compound which is at least about 85%, such as at least about 90%, at least about 95%, at least about 98%, at least about 99%, at least about 99.5% or at least about 99.9%, as measured by a HPLC.

As used herein, the terms “amorphous” or “stable amorphous” form of the compound of Formula I are interchangeable and an “amorphous” compound of Formula I indicates that the compound of Formula I is present in substantially amorphous state. “Substantially pure amorphous” denotes that at least about 90%, such at least about 95%, at least about 98%, at least about 99% or at least about 99.5% of the compound of Formula I is amorphous in nature.

As used herein, in some embodiments, the term “suitable solvent” or “solvent” includes solvents that may be used for preparing a compound of Formula I or a pharmaceutically acceptable salt thereof (and its intermediates) and may be selected from, but not limited to, C₁-C₆ alcohols, C₁-C₈ hydrocarbons, halogenated hydrocarbons, ethers, C₃-C₈ ketones, esters, nitriles, sulphonamides, acetamides, pyrrolidines, formamides, water and mixture of any of the foregoing. Examples include, but are not limited to methanol, ethanol, butanol, t-butanol, isopropyl alcohol, n-propyl alcohol, iso-butanol, pentanol, glycols, toluene, chlorobenzene, acetonitrile, dimethyl acetamide (DMA), dimethylformamide (DMF), N-methyl pyrrolidine (NMP), dimethyl sulfoxide (DMSO), hexamethyl phosphoramide (HMPA), tetrahydrofuran (THF), methyl tetrahydrofuran, dioxane, acetone, methyl ethyl ketone (MEK), methyl isobutyl ketone (MIBK), methyl t-butyl ketone, dichloromethane, dichloroethane, chloroform, tetrachloromethane, chlorobenzene, ethyl acetate, propyl acetate, propenyl acetate, t-butyl acetate, hexane, n-heptane, cyclohexane, petroleum benzine, water and any combination of any of the foregoing.

The present invention will now be explained in more detail. While the invention is susceptible to various modifications and alternative forms, specific embodiments thereof are described in detail below. It should be understood, however that it is not intended to limit the invention to the particular forms disclosed, but on the contrary, the invention is to cover all modifications, equivalents, and alternatives falling within the scope of the invention.

The compound of Formula I is also known as 2-((S)-1-(1-(5-ethyl-pyrimidin-2-yl) piperidin-4-yl) ethoxy)-6-(2-fluoro-4-(methylsulfonyl)phenyl) imidazo[2,1-b][1,3,4]thiadiazole.

### Crystalline Form M-1

In one embodiment, the present invention relates to a crystalline Form M-1 of the compound of Formula I, characterized by an XRPD pattern exhibiting one or more (such as 2 or 3) characteristic peaks at 6.0±0.2, 8.1±0.2 and 17.3±0.2° 2θ. In one embodiment, Form M-1 further exhibits characteristic XRPD peaks at 4.5±0.2, 7.0±0.2, 10.5±0.2, 12.5±0.2, 14.9±0.2, 15.1±0.2, 15.4±0.2, 16.6±0.2, 18.6±0.2, 19.3±0.2, 20.4±0.2, 25.8±0.2 and 26.4±0.2° 2θ.

In one embodiment, the present invention relates to a crystalline Form M-1 of the compound of Formula I, characterized by an XRPD pattern exhibiting one or more (such as 2, 3, 4, 5, 6, 7 or 8) characteristic peaks at 4.5±0.2, 6.0±0.2, 8.1±0.2, 14.9±0.2, 15.1±0.2, 15.4±0.2, 17.3±0.2 and 18.6±0.2° 20 and, optionally, one or more additional peaks at 7.0±0.2, 10.5±0.2, 12.5±0.2, 16.6±0.2, 19.3±0.2, 20.4±0.2, 25.8±0.2 and 26.4±0.2° 2θ.

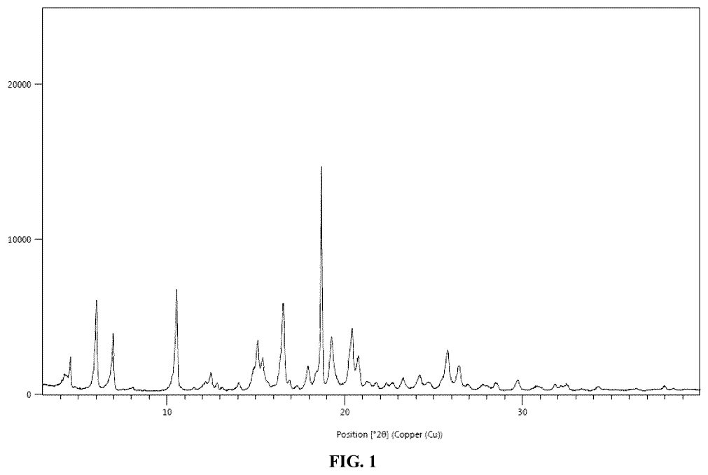

In another embodiment, the present invention relates to a crystalline Form M-1 of the compound of Formula 1 characterized by an XRPD pattern substantially as depicted in FIG. 1.

In another embodiment, the present invention relates to a crystalline Form M-1 of the compound of Formula I characterized by a DSC plot exhibiting endothermic peaks at about 111° C. and about 141.7° C.

In another embodiment, the present invention relates to a crystalline Form M-1 of the compound of Formula I characterized by a thermogravimetric analysis (TGA) thermogram that exhibits a mass loss of about 0.01 to about 0.20±1% when heated to about 110° C.

In another embodiment, the present invention relates to a process for the preparation of Form M-1 of compound of Formula I, comprising,
a) mixing a compound of Formula I in one or more solvents (e.g., acetonitrile);
b) heating the reaction mixture (e.g., from about 40° C. to reflux temperature);
c) cooling the reaction mixture (e.g., to room temperature);
d) optionally seeding the reaction mixture with crystalline Form M-1 of the compound of Formula I; and
e) optionally isolating the crystalline Form M-1 of the compound of Formula I.

In one embodiment, the one or more solvents used in step a) is selected from methanol, ethanol, 1-propanol, 2-propanol, n-butanol, isobutanol, nitromethane, chloroform, acetonitrile, acetone, Methyl isobutyl ketone (MIBK), methyl ethyl ketone (MEK), toluene, heptane, ethyl acetate, propyl acetate, n-pentyl acetate, isopropyl acetate, butyl acetate, propionitrile, diethylether, dimethylether, diisopropylether, diphenylether, Methyl tert-butyl ether (MTBE), tetrahydrofuran, methyl tetrahydrofuran, 1,4-dioxane, dimethoxyethane, o-xylene, m-xylene, p-xylene, n-pentane, cyclopentane, n-hexane, cyclohexane, benzene, dichloromethane, dichloroethane, tetrachloromethane, chlorobenzene, dimethyl acetamide (DMA), dimethylformamide (DMF), water and any mixture of any of the foregoing.

In one embodiment, the solvent used in step a) is selected from acetonitrile, methanol, and a mixture thereof.

In one embodiment the present invention relates to a process for the preparation of crystalline Form M-1 of the compound of Formula I, the process comprising,
a) mixing a compound of Formula I and acetonitrile;
b) heating the reaction mixture to between about 60° C. and reflux temperature (e.g., about 179° C.);
c) cooling the reaction mixture to room temperature;
d) optionally seeding the reaction mixture with crystalline Form M-1, optionally at a temperature of about 30° C. to about 40° C.; and

e) optionally isolating the crystalline Form M-1 of the compound of Formula I.

In another embodiment, the present invention relates to crystalline form M-1 of the compound of Formula I having a particle size distribution characterized by a d₉₀ of from about 60 μm to about 100 μm.

### Crystalline Form M-2

In another embodiment, the present invention relates a crystalline Form M-2 of the compound of Formula I, characterized by an XRPD pattern exhibiting one or more (such as 2, 3, 4, 5, 6, 7 or 8) characteristic peaks at 6.9±0.2, 10.6±0.2, 16.6±0.2, 19.2±0.2 and 20.4±0.2° 2θ.

In another embodiment, Form M-2 further exhibits on or more characteristic XRPD peaks at 4.5±0.2, 12.5±0.2, 14.1±0.2, 14.9±0.2, 15.4±0.2, 17.9±0.2 and 20.8±0.2° 2θ.

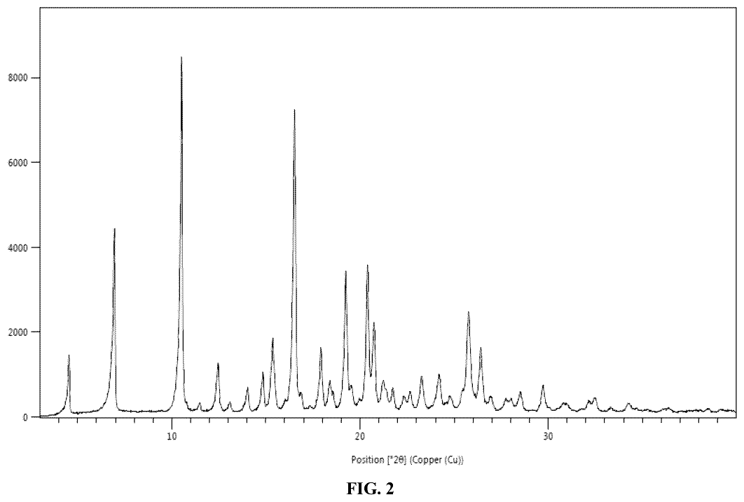

In another embodiment, the present invention relates to a crystalline Form M-2 of the compound of Formula 1 characterized by an XRPD pattern substantially as depicted in FIG. 2.

In another embodiment, the present invention relates to a crystalline Form M-2 of compound of Formula I characterized by a DSC exhibiting an endothermic peak at about 144° C.

In another embodiment, the present invention relates to a crystalline Form M-2 of compound of Formula I, characterized by a DSC exhibiting an onset at about 140° C. and an endothermic peak at about 144° C.

In another embodiment, the present invention relates to a crystalline Form M-2 of the compound of Formula I characterized by dynamic vapour sorption analysis showing a weight gain of not more than 0.1%, such as not more than 0.05% or not more than 0.01%, when the crystalline Form M-2 is equilibrated at 25° C.±1° C. and 80%+2% RH for 24 hours.

In another embodiment, the present invention relates to a crystalline Form M-2 of the compound of Formula I characterized by a thermogravimetric analysis (TGA) thermogram that exhibits a mass loss of about 0.01 to about 0.10±1% when heated to about 110° C.

In another embodiment, the present invention relates to crystalline form M-2 of the compound of Formula I having a particle size distribution characterized by a d₉₀ of from about 60 μm to about 100 μm.

In another embodiment, the present invention relates to a crystalline Form M-2 of the compound of formula I that is non-hygroscopic.

In another embodiment the present invention relates to a process for the preparation of crystalline Form M-2 of the compound of Formula I, the process comprising,
a) mixing racemic compound of Formula I with one or more solvents;
b) stirring the reaction mixture (e.g., for about 1-2 hrs at about 20-40° C.);
c) isolating the resulting solid (e.g., by filtering the reaction mixture);
d) obtaining the desired isomer of the of crystalline Form M-2 of the compound of Formula I (e.g., performing a chiral resolution using chiral chromatography); and
e) optionally isolating the crystalline Form M-2 of the compound of Formula I.

In another embodiment the present invention provides a process for the preparation of Form M-2 of compound of Formula I, comprising,
a) mixing racemic compound of Formula I with one or more solvents;
b) stirring the reaction mixture (e.g., for about 1-2 hrs at about 20-40° C.);
c) isolating the resulting solid (e.g., by filtering the reaction mixture;
d) obtaining the desired isomer of the of crystalline Form M-2 of the compound of Formula I by chiral chromatography using a mixture of dichloromethane/methanol (1:1); and
e) optionally isolating the crystalline Form M-2 of the compound of Formula I.

In another embodiment the present invention provides a process for the preparation of crystalline Form M-2 of the compound of Formula I, comprising,
a) mixing racemic compound of Formula I with methanol;
b) stirring the reaction mixture (e.g., for about 1-2 hrs at about 20-40° C.);
c) isolating the resulting solid (e.g., by filtering the reaction mixture);
d) obtaining the desired isomer of the of crystalline Form M-2 of the compound of Formula I by chiral chromatography using a mixture of dichloromethane/methanol (1:1); and
e) optionally isolating the crystalline Form M-2 of the compound of Formula I.

In one embodiment, the one or more solvents used in step a) is selected from methanol, ethanol, 1-propanol, 2-propanol, n-butanol, isobutanol, nitromethane, chloroform, acetonitrile, acetone, MIBK, MEK, toluene, heptane, ethyl acetate, propyl acetate, n-pentyl acetate, isopropyl acetate, butyl acetate, propionitrile, diethylether, dimethylether, diisopropylether, diphenylether, MTBE, tetrahydrofuran, methyl tetrahydrofuran, 1,4-dioxane, dimethoxyethane, o-xylene, m-xylene, p-xylene, n-pentane, cyclopentane, n-hexane, cyclohexane, benzene, dichloromethane, dichloroethane, tetrachloromethane, chlorobenzene, dimethyl acetamide (DMA), dimethylformamide (DMF), water, and any mixture of any of the foregoing.

In one embodiment, the solvent used in step a) is methanol.

In another embodiment the present invention provides a process for the preparation of crystalline Form M-2 of the compound of Formula I, the process comprising heating a compound of Formula I (such as amorphous or crystalline Form M-1) to give crystalline Form M-2. In one embodiment, the compound of Formula I (such as amorphous or crystalline Form M-1) is heated for example to a temperature between about 100 and 130° C. for about 1-4 hrs to give crystalline Form M-2.

### Crystalline Form M-3

In another embodiment, the present invention relates to a crystalline Form M-3 of the compound of Formula I, characterized by an XRPD pattern exhibiting one or more (such as 2, 3, or 4) characteristic peaks at about 7.1±0.2, 13.3±0.2, 17.6±0.2 and 21.8±0.2° 2θ.

In another embodiment, crystalline Form M-3 further comprises one or more characteristic XRPD pattern at 4.3±0.2, 11.1±0.2, 15.2±0.2, 15.7±0.2, 16.0±0.2, 17.5±0.2, 18.8±0.2, 20.2±0.2, 21.7±0.2, 23.2±0.2 and 25.7±0.2° 2θ.

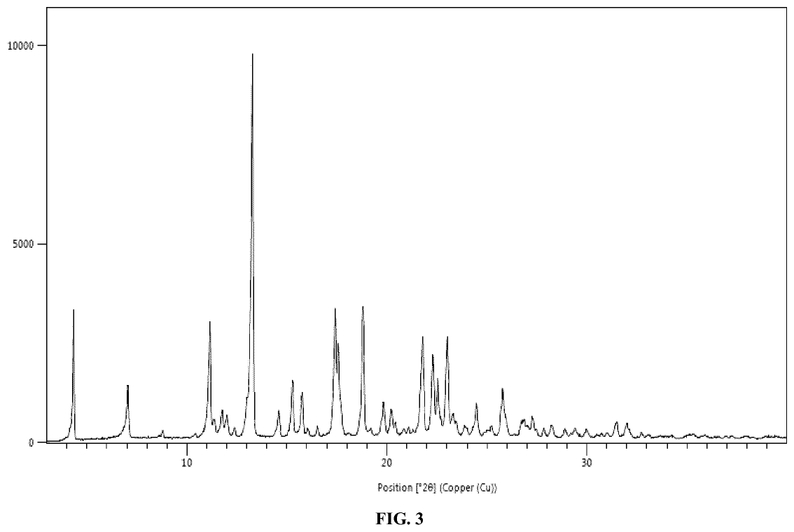

In another embodiment, the present invention relates to a crystalline Form M-3 of the compound of Formula 1 characterized by an XRPD pattern substantially as depicted in FIG. 3.

In another embodiment, the present invention relates to a crystalline Form M-3 of the compound of Formula I characterized by a DSC exhibiting an endothermic peak at about 113° C.

In another embodiment, the present invention relates to a crystalline Form M-3 of the compound of Formula I characterized by a DSC exhibiting an onset at about 109° C. and an endothermic peak at about 113° C.

In another embodiment, the present invention relates to a crystalline Form M-3 of the compound of Formula I characterized by a thermogravimetric analysis (TGA) thermogram that exhibits a mass loss of about 4.7 to about 5.7±1% when heated to about 140° C.

In another embodiment, the present invention relates to crystalline form M-3 of the compound of Formula I having a particle size distribution characterized by a doo of from about 60 μm to about 100 μm.

In another embodiment, the present invention provides a process for the preparation of crystalline Form M-3 of the compound of Formula I, the process comprising:
a) dissolving a compound of Formula I with one or more solvents;
b) heating the reaction mixture; and
c) isolating the crystalline Form M-3 of the compound of Formula I.

In another embodiment, the present invention relates to a process for the preparation of crystalline Form M-3 of the compound of Formula I, the process comprising:
a) mixing a compound of Formula I with one or more solvents;
b) stirring the reaction mass at ambient temperature for about 10-15 hrs; and
c) isolating the crystalline Form M-3 of the compound of Formula I.

In one embodiment, the compound of Formula I used in step a) is selected from amorphous, one or more crystalline forms or any mixture of any of the foregoing. In one embodiment, the compound of Formula I used in step a) is crystalline form M-1.

In another embodiment the one or more solvents used in step a) is selected from ethanol, methanol, 1-propanol, 2-propanol, n-butanol, isobutanol, nitromethane, chloroform, acetonitrile, acetone, MIBK, MEK, toluene, heptane, ethyl acetate, propyl acetate, n-pentyl acetate, isopropyl acetate, butyl acetate, propionitrile, diethylether, dimethylether, diisopropylether, diphenylether, MTBE, tetrahydrofuran, methyl tetrahydrofuran, 1,4-dioxane, dimethoxyethane, o-xylene, m-xylene, p-xylene, n-pentane, cyclopentane, n-hexane, cyclohexane, heptane, benzene, dichloromethane, dichloroethane, tetrachloromethane, chlorobenzene, dimethyl acetamide (DMA), dimethylformamide (DMF), water and any mixture of any of the foregoing.

In preferred embodiment, the one or more solvents used in step a) is selected from ethanol, heptane and a mixture thereof.

### Crystalline Form M-4

In another embodiment, the present invention relates to a crystalline Form M-4 of the compound of Formula I characterized by an XRPD pattern exhibiting characteristic peaks at 8.8±0.2, 11.3±0.2, 17.8±0.2, 19.0±0.2, 23.1±0.2 and 25.8±0.2° 2θ;

In another embodiment, the present invention relates a crystalline Form M-4 of the compound of Formula I, characterized by an XPRD pattern exhibiting characteristic peaks at 7.2±0.2, 8.8±0.2, 13.3±0.2, 14.7±0.2, 19.4±0.2, 24.5±0.2, 25.9±0.2 and 27.3±0.2° 2θ, and one or more (such as 2, 3, 4, 5, 6, 7, or 8) additional characteristic peaks at 11.5±0.2, 12.3±0.2, 15.6±0.2, 16.0±0.2, 17.8±0.2, 19.0±0.2, 22.0±0.2 and 23.1±0.2° 2θ.

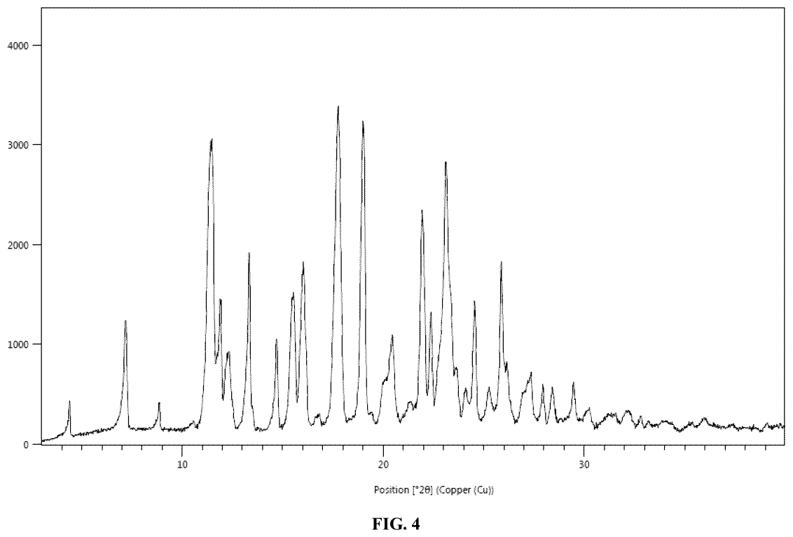

In another embodiment, the present invention relates to a crystalline Form M-4 of the compound of Formula 1 characterized by an XRPD pattern substantially as depicted in FIG. 4.

In another embodiment, the present invention provides a crystalline Form M-4 of the compound of Formula I, characterized by DSC exhibiting an endothermic peak at about 107° C.

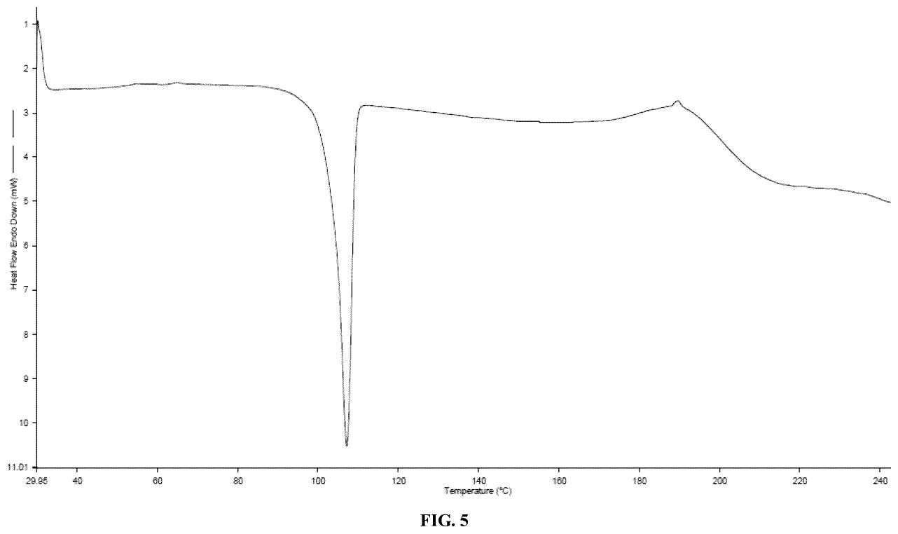

In another embodiment, the present invention provides a crystalline Form M-4 of the compound of Formula I, characterized by a DSC substantially as depicted in FIG. 5.

In another embodiment, the present invention relates to a crystalline Form M-4 of the compound of Formula I characterized by a thermogravimetric analysis (TGA) thermogram that exhibits a mass loss of about 0.1 to about 0.8±1% when heated to about 110° C.

In another embodiment, the present invention relates to crystalline form M-4 of the compound of Formula I having a particle size distribution characterized by a d₉₀ of from about 60 μm to about 100 μm.

In another embodiment the present invention relates to a process for the preparation of crystalline Form M-4 of compound of Formula I, comprising,
a) mixing a compound of Formula I and one or more solvent(s) (e.g., a combination of ethanol and heptane);
b) heating the reaction mixture (e.g., to a suitable temperature for a suitable time);
c) crystallizing the reaction mixture; and
d) optionally isolating the crystalline Form M-4 of the compound of Formula I.

In another embodiment the present invention relates to a process for the preparation of crystalline Form M-4 of compound of Formula I, comprising,
a) adding a compound of Formula I to one or more solvents;
b) stirring the reaction mixture (e.g., for about 20 to about 60 minutes); and
c) optionally isolating the crystalline Form M-4 of the compound of Formula I.

In one embodiment, the compound of Formula I used in step a) of a process for the preparation of Form M-4 is selected from amorphous, one or more crystalline form and any mixture thereof. In one embodiment, the compound of Formula I used in step a) of the process for the preparation of Form M-4 is crystalline Form M-1.

In another embodiment the present invention relates to a process for the preparation of crystalline Form M-4 of the compound of Formula I, the process comprising heating crystalline Form M-1 of the compound of Formula I (e.g., to a suitable temperature for a suitable time) to give crystalline Form M-4 of the compound of Formula I.

In another embodiment the present invention relates to a process for the preparation of crystalline Form M-4 of the compound of Formula I, the process comprising heating crystalline Form M-1 of the compound of Formula I to about 50 to about 70° C. for about 70 to about 75 hrs to give crystalline Form M-4 of the compound of Formula I.

In another embodiment the present invention relates to a process for the preparation of crystalline Form M-3 of the compound of Formula I, the process comprising heating crystalline Form M-1 of the compound of Formula I (e.g., to a suitable temperature for a suitable time) to give crystalline Form M-4 of the compound of Formula I.

In another embodiment the present invention relates to a process for the preparation of crystalline Form M-4 of the compound of Formula I, the process comprising heating crystalline Form M-3 of the compound of Formula I to about 50 to about 70° C. for about 70 to about 75 hrs to give crystalline Form M-4 of the compound of Formula I.

In certain embodiments, the one or more solvent used in step a) of a process for the preparation of Form M-4 is selected from ethanol, methanol, 1-propanol, 2-propanol, n-butanol, isobutanol, nitromethane, heptane, chloroform, acetonitrile, acetone, MIBK, MEK, toluene, ethyl acetate, propyl acetate, n-pentyl acetate, isopropyl acetate, butyl acetate, propionitrile, diethylether, dimethylether, diisopropylether, diphenylether, MTBE, tetrahydrofuran, methyl tetrahydrofuran, 1,4-dioxane, dimethoxyethane, o-xylene, m-xylene, p-xylene, n-pentane, cyclopentane, n-hexane, cyclohexane, benzene, dichloromethane, dichloroethane, tetrachloromethane, chlorobenzene, dimethyl acetamide (DMA), dimethylformamide (DMF), water and any mixture thereof.

In one embodiment, the one or more solvents used in step a) is selected from methanol, ethanol, acetonitrile, heptane, and any mixture thereof.

### Crystalline Form M-5

In another embodiment, the present invention relates to a crystalline Form M-5 of the compound of Formula I characterized by an XPRD pattern exhibiting characteristic peaks at about 4.3±0.2, 7.8±0.2, 10.2±0.2, 16.3±0.2 and 16.8±0.2° 2θ.

In another embodiment, the present invention relates to a crystalline Form M-5 of the compound of Formula I characterized by an XPRD pattern exhibiting characteristic peaks at about 4.3±0.2, 7.6±0.2, 16.3±0.2 and 16.8±0.2° 20 and one or more (such as 2, 3 or 4) additional peaks at 12.3±0.2, 17.8±0.2, 19.4±0.2 and 20.6±0.2° 2θ.

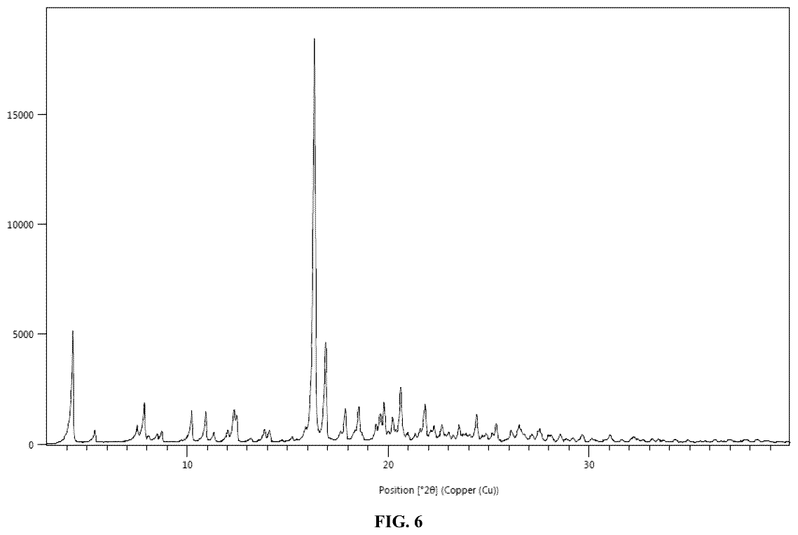

In another embodiment, the present invention relates to a crystalline Form M-5 of the compound of Formula 1 characterized by an XRPD pattern substantially as depicted in FIG. 6.

In another embodiment, the present invention relates to a crystalline Form M-5 of the compound of Formula I characterized by a DSC exhibiting an onset at about 146° C. and an endothermic peak at about 148° C.

In another embodiment, the present invention relates to a crystalline Form M-5 of the compound of Formula I characterized by a DSC exhibiting an endothermic peak at about 148° C.

In another embodiment, the present invention relates to a crystalline Form M-5 of the compound of Formula I characterized by a thermogravimetric analysis (TGA) thermogram that exhibits a mass loss of about 0.03 to about 0.1±1% when heated to about 110° C.

In another embodiment, the present invention relates to crystalline form M-5 of the compound of Formula I having a particle size distribution characterized by a doo of from about 60 μm to about 100 μm.

In another embodiment the present invention relates to a process for the preparation of crystalline Form M-5 of the compound of Formula I, the process comprising,
a) mixing a compound of Formula I in one or more solvents;
b) stirring the reaction mass (e.g., at ambient temperature for about 10-15 hrs); and
c) optionally isolating the crystalline Form M-5 of the compound of Formula I.

In another embodiment the present invention relates to a process for the preparation of crystalline Form M-5 of the compound of Formula I, the process comprising,
a) mixing a compound of Formula I in acetonitrile;
b) stirring the reaction mass at ambient temperature for about 0-15 hrs; and
c) optionally isolating the crystalline Form M-5 of the compound of Formula I.

In another embodiment, the compound of Formula I used in step a) of the process for the preparation of crystalline Form M-5 of the compound of Formula I selected from amorphous, one or more crystalline forms, or any mixture of any of the foregoing. In one embodiment, the compound of Formula I used in step a) of the process for the preparation of crystalline Form M-5 of the compound of Formula I crystalline form M-2.

In another embodiment the one or more solvents used in step a) of the process for the preparation of crystalline Form M-5 of the compound of Formula I is selected from acetonitrile, methanol, ethanol, 1-propanol, 2-propanol, n-butanol, isobutanol, nitromethane, chloroform, acetone, MIBK, MEK, toluene, heptane, ethyl acetate, propyl acetate, n-pentyl acetate, isopropyl acetate, butyl acetate, propionitrile, diethylether, dimethylether, diisopropylether, diphenylether, MTBE, tetrahydrofuran, methyl tetrahydrofuran, 1,4-dioxane, dimethoxyethane, o-xylene, m-xylene, p-xylene, n-pentane, cyclopentane, n-hexane, cyclohexane, benzene, dichloromethane, dichloroethane, tetrachloromethane, chlorobenzene, dimethyl acetamide (DMA), dimethylformamide (DMF), water, and any mixture thereof.

In one embodiment, the one or more solvents used in step a) of the process for the preparation of crystalline Form M-5 of the compound of Formula I is selected from nitromethane, acetonitrile, and a mixture thereof.

### Crystalline Form M-6

In another embodiment, the present invention relates to a crystalline Form M-6 of the compound of Formula I characterized by an XPRD pattern exhibiting characteristic peaks at 5.6±0.2, 9.7±0.2, and 19.8±0.2° 2θ.

In another embodiment, the present invention relates to a crystalline Form M-6 of the compound of Formula I characterized by an XPRD pattern exhibiting characteristic peaks at 5.6±0.2, 9.7±0.2 and 19.8±0.2° 20 and one or more additional peak selected from 15.9±0.2, 17.3±0.2, 18.6±0.2 and 20.3±0.2° 2θ.

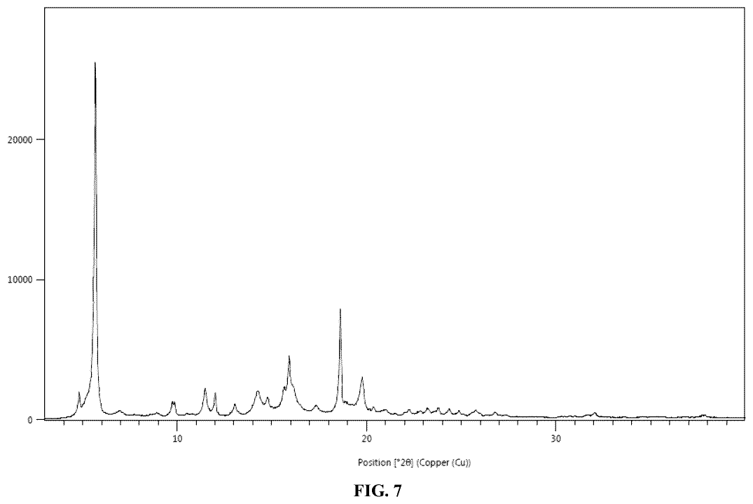

In another embodiment, the present invention provides a crystalline Form M-6 of the compound of Formula 1 characterized by an XRPD pattern substantially as depicted in FIG. 7.

In another embodiment, the present invention provides a crystalline Form M-6 of the compound of Formula I characterized by a DSC exhibiting onset and endothermic peaks at about 80° C. and about 87° C., about 103° C. and about 112° C., and 139° C. and about 142° C., respectively.

In another embodiment, the present invention provides a crystalline Form M-6 of the compound of Formula I characterized by a DSC exhibiting endothermic peaks at about 87° C., about 112° C. and about 142° C.

In another embodiment, the present invention relates to a crystalline Form M-6 of the compound of Formula I characterized by a thermogravimetric analysis (TGA) thermogram that exhibits a mass loss of about 0.3 to about 1.5±1% when heated to about 120° C.

In another embodiment, the present invention relates to crystalline form M-6 of the compound of Formula I having a particle size distribution characterized by a d₉₀ of from about 60 μm to about 100 μm.

In another embodiment the present invention relates a process for the preparation of Form M-6 of the compound of Formula I, the process comprising:
a) adding compound of Formula I to one or more solvents under liquid nitrogen to afford a solid mass;
b) maintaining the solid mass at room temperature to afford a clear solution; and
c) optionally isolating the crystalline Form M-6 of the compound of Formula I.

In another embodiment, the compound of Formula I used in step a) of the process for the preparation of crystalline Form M-6 of the compound of Formula I is selected from amorphous, one or more crystalline forms, and any mixture thereof. In another embodiment, the compound of Formula I used in step a) of the process for the preparation of crystalline Form M-6 of the compound of Formula I is crystalline form M-2.

In another embodiment the one or more solvents used in step a) of the process for the preparation of crystalline Form M-6 of the compound of Formula I is selected from nitromethane, methanol, ethanol, 1-propanol, 2-propanol, n-butanol, isobutanol, chloroform, acetonitrile, acetone, MIBK, MEK, toluene, heptane, ethyl acetate, propyl acetate, n-pentyl acetate, isopropyl acetate, butyl acetate, propionitrile, diethylether, dimethylether, diisopropylether, diphenylether, MTBE, tetrahydrofuran, methyl tetrahydrofuran, 1,4-dioxane, dimethoxyethane, o-xylene, m-xylene, p-xylene, n-pentane, cyclopentane, n-hexane, cyclohexane, benzene, dichloromethane, dichloroethane, tetrachloromethane, chlorobenzene, dimethyl acetamide (DMA), dimethylformamide (DMF), water, and any mixture thereof.

In one embodiment, the one or more solvents used in step a) of the process for the preparation of crystalline Form M-6 of the compound of Formula I is selected from o-xylene, heptane, nitromethane, and any mixture thereof.

### Crystalline Form M-7

In another embodiment, the present invention relates to a crystalline Form M-7 of the compound of Formula I characterized by an XRPD pattern exhibiting characteristic peaks at 4.3±0.2, 8.7±0.2, 10.6±0.2, 16.4±0.2, 18.5±0.2, 19.5±0.2 and 21.9±0.2° 2θ.

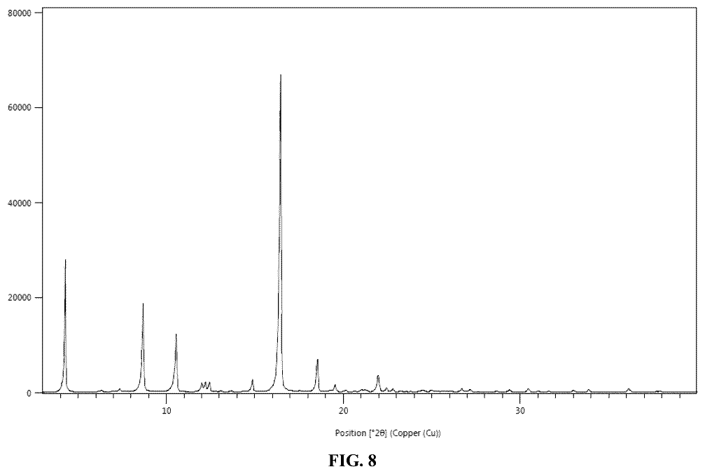

In another embodiment, the present invention relates to a crystalline Form M-7 of the compound of Formula 1 characterized by an XRPD pattern substantially as depicted in FIG. 8.

In another embodiment, the present invention relates to a crystalline Form M-7 of the compound of Formula I characterized by a DSC exhibiting an onset peak at about 97° C. and an endothermic peak at about 100° C.

In another embodiment, the present invention relates to a crystalline Form M-7 of the compound of Formula I characterized by a DSC exhibiting an endothermic peak at about 100° C.

In another embodiment, the present invention relates to a crystalline Form M-7 of the compound of Formula I characterized by a thermogravimetric analysis (TGA) thermogram that exhibits a mass loss of about 5.0 to about 6.0±1% when heated to about 150° C.

In another embodiment, the present invention relates to crystalline form M-7 of the compound of Formula I having a particle size distribution characterized by a doo of from about 60 μm to about 100 μm.

In another embodiment the present invention relates to a process for the preparation of Form M-7 of the compound of Formula I, the process comprising:
a) mixing a compound of Formula I in one or more solvents;
b) heating the reaction mixture obtained in step a);
c) evaporating the solvent; and
d) optionally isolating the crystalline Form M-7 of the compound of Formula I.

In another embodiment, the compound of Formula I used in step a) of the process for the preparation of crystalline Form M-7 of the compound of Formula I is selected from amorphous, one or more crystalline forms, and any mixture thereof. In another embodiment, the compound of Formula I used in step a) of the process for the preparation of crystalline Form M-7 of the compound of Formula I is crystalline form M-2.

In another embodiment, the one or more solvents used in step a) of the process for the preparation of crystalline Form M-7 of the compound of Formula I is selected from o-xylene, m-xylene, p-xylene, heptane, methanol, ethanol, 1-propanol, 2-propanol, n-butanol, isobutanol, nitromethane, chloroform, acetonitrile, acetone, MIBK, MEK, toluene, ethyl acetate, propyl acetate, n-pentyl acetate, isopropyl acetate, butyl acetate, propionitrile, diethylether, dimethylether, diisopropylether, diphenylether, MTBE, tetrahydrofuran, methyl tetrahydrofuran, 1,4-dioxane, dimethoxyethane, cyclopentane, n-hexane, cyclohexane, benzene, dichloromethane, dichloroethane, tetrachloromethane, chlorobenzene, dimethyl acetamide (DMA), dimethylformamide (DMF), water, and any mixture thereof.

In one embodiment, the one or more solvent used in step a) of the process for the preparation of crystalline Form M-7 of the compound of Formula I is selected from 2-propanol, hexane, o-xylene, heptane, and mixture thereof.

### Crystalline Form M-8

In another embodiment, the present invention provides a crystalline Form M-8 of the compound of Formula I characterized by an XRPD pattern exhibiting characteristic peaks at 4.6±0.2, 6.4±0.2, 9.3±0.2, 12.3±0.2, 15.5±0.2, 17.0±0.2 and 17.7±0.2° 2θ.

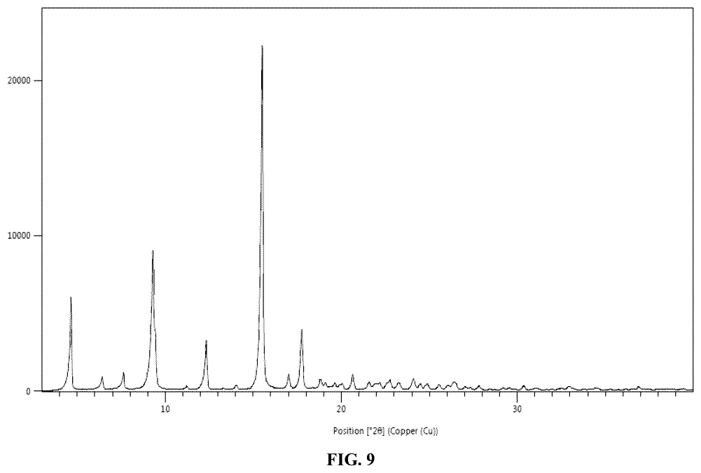

In another embodiment, the present invention relates to a crystalline Form M-8 of the compound of Formula 1 characterized by an XRPD pattern substantially as depicted in FIG. 9.

In another embodiment, the present invention provides a crystalline Form M-8 of compound of Formula I characterized by a DSC exhibiting an onset at about 127° C. and an endothermic peak at about 133° C.

In another embodiment, the present invention provides a crystalline Form M-8 of compound of Formula I characterized by a DSC exhibiting an endothermic peak at about 133° C.

In another embodiment, the present invention relates to a crystalline Form M-8 of the compound of Formula I characterized by a thermogravimetric analysis (TGA) thermogram that exhibits a mass loss of about 0.05 to about 0.5±1% when heated to about 110° C.

In another embodiment, the present invention relates to crystalline form M-8 of the compound of Formula I having a particle size distribution characterized by a d₉₀ of from about 60 μm to about 100 μm.

In another embodiment the present invention relates to a process for the preparation of crystalline Form M-8 of the compound of Formula I, the process comprising:
a) mixing a compound of Formula I in one or more solvents;
b) heating the reaction mixture of step a);
c) cooling the reaction mixture obtained from step b); and
d) optionally isolating the crystalline Form M-8 of the compound of Formula I.

In another embodiment, the compound of Formula I used in step a) of the process for the preparation of crystalline Form M-8 of the compound of Formula I is selected from amorphous, one or more crystalline forms, and any mixture thereof. In another embodiment, the compound of Formula I used in step a) of the process for the preparation of crystalline Form M-8 of the compound of Formula I is crystalline form M-1.

In another embodiment the one or more solvents used in step a) of the process for the preparation of crystalline Form M-8 of the compound of Formula I is selected from isopropanol, hexane, heptane, methanol, ethanol, 2-propanol, n-butanol, isobutanol, nitromethane, chloroform, acetonitrile, acetone, MIBK, MEK, toluene, ethyl acetate, propyl acetate, n-pentyl acetate, isopropyl acetate, butyl acetate, propionitrile, diethylether, dimethylether, diisopropylether, diphenylether, MTBE, tetrahydrofuran, methyl tetrahydrofuran, 1,4-dioxane, dimethoxyethane, o-xylene, m-xylene, p-xylene, n-pentane, cyclopentane, cyclohexane, benzene, dichloromethane, dichloroethane, tetrachloromethane, chlorobenzene, dimethyl acetamide (DMA), dimethylformamide (DMF), water, and any mixture thereof.

In one embodiment, the one or more solvents used in step a) of the process for the preparation of crystalline Form M-8 of the compound of Formula I is selected from methanol, isopropanol, hexane, and any mixture thereof.

### Crystalline Form M-9

In another embodiment, the present invention relates to a crystalline Form M-9 of compound of Formula I characterized by an XRPD pattern exhibiting characteristic peaks at 7.2±0.2, 8.8±0.2, 11.5±0.2, 12.3±0.2, 13.3±0.2, 15.6±0.2, 16.0±0.2, 17.9±0.2, 19.0±0.2, 22.3±0.2, 23.2±0.2, 25.8±0.2, 27.3±0.2 and 29.4±0.2° 2θ.

In another embodiment, the present invention relates to a crystalline Form M-9 of compound of Formula I characterized by an XRPD pattern exhibiting characteristic peaks at 4.3±0.2, 8.8±0.2, 11.5±0.2, 12.3±0.2, 13.2±0.2, 15.5±0.2, 16.0±0.2, 17.9±0.2, 19.0±0.2, 22.3±0.2, 23.2±0.2, 25.8±0.2, 27.3±0.2 and 29.4±0.2° 20 and one or more (such as 2, 3, 4, 5, 6, 7, 8 or 9) additional peaks selected from 11.7±0.2, 11.9±0.2, 14.7±0.2, 16.5±0.2, 20.5±0.2, 21.3±0.2, 24.5±0.2, 26.3±0.2 and 28.4±0.2° 2θ.

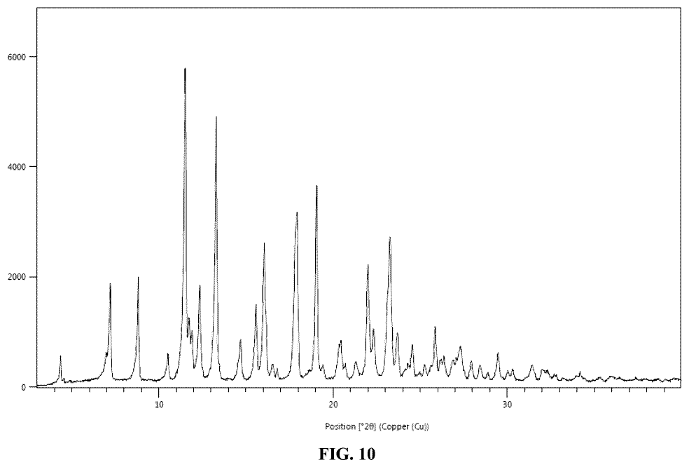

In another embodiment, the present invention relates to a crystalline Form M-9 of compound of Formula 1 characterized by ab XRPD pattern substantially as depicted in FIG. 10.

In another embodiment, the present invention relates to a crystalline Form M-9 of the compound of Formula I characterized by a DSC exhibiting two onset and endothermic peaks at about 98° C. and about 108° C., and at about 138° C. and about 142° C. respectively.

In another embodiment, the present invention relates to a crystalline Form M-9 of the compound of Formula I characterized by a DSC exhibiting endothermic peaks at about 108° C. and about 142° C.

In another embodiment, the present invention relates to a crystalline Form M-9 of the compound of Formula I characterized by a thermogravimetric analysis (TGA) thermogram that exhibits a mass loss of about 0.07 to about 0.5±1% when heated to about 110° C.

In another embodiment, the present invention relates to crystalline form M-9 of the compound of Formula I having a particle size distribution characterized by a d₉₀ of from about 60 μm to about 100 μm.

In another embodiment the present invention relates to a process for the preparation of crystalline Form M-9 of the compound of Formula I, the process comprising:
a) mixing a compound of Formula I in one or more solvents under liquid nitrogen to afford a solid mass;
b) maintaining the solid mass at room temperature to afford a clear solution;
c) optionally isolating the crystalline Form M-9 of the compound of Formula I.

In another embodiment, the compound of Formula I used in step a) of the process for the preparation of crystalline Form M-9 of the compound of Formula I is selected from amorphous, one or more crystalline forms, and any mixture thereof. In another embodiment, the compound of Formula I used in step a) of the process for the preparation of crystalline Form M-9 of the compound of Formula I is crystalline form M-2.

In another embodiment the one or more solvents used in step a) of the process for the preparation of crystalline Form M-9 of the compound of Formula I is selected from methanol, ethanol, isopropanol, 2-propanol, n-butanol, isobutanol, nitromethane, chloroform, acetonitrile, acetone, MIBK, MEK, toluene, heptane, ethyl acetate, propyl acetate, n-pentyl acetate, isopropyl acetate, butyl acetate, propionitrile, diethylether, dimethylether, diisopropylether, diphenylether, MTBE, tetrahydrofuran, methyl tetrahydrofuran, 1,4-dioxane, dimethoxyethane, o-xylene, m-xylene, p-xylene, n-pentane, cyclopentane, n-hexane, cyclohexane, benzene, dichloromethane, dichloroethane, tetrachloromethane, chlorobenzene, dimethyl acetamide (DMA), dimethylformamide (DMF), water, and any mixture thereof.

In another embodiment the one or more solvents used in step a) of the process for the preparation of crystalline Form M-9 of the compound of Formula I selected from acetonitrile, water, methanol, and any mixture thereof.

### Amorphous Form

In another embodiment, the present invention relates to an amorphous form of the compound of Formula I and a process for its preparation.

In another embodiment, the present invention relates to a stable amorphous form of the compound of Formula I and a process for its preparation.

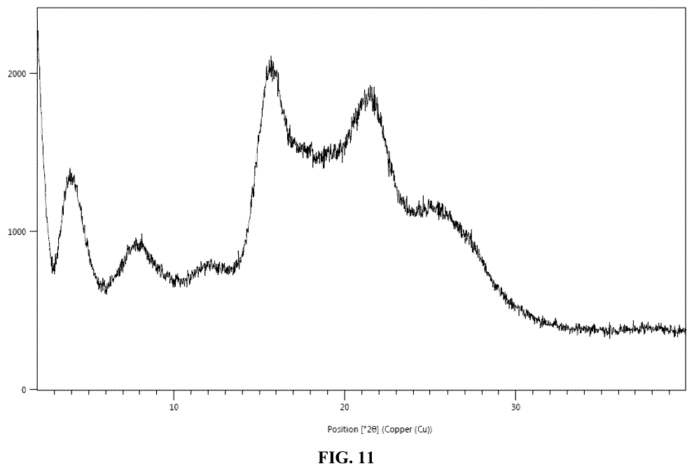

In another embodiment, the present invention relates to a stable amorphous form of the compound of Formula I characterized by an XRPD pattern substantially as depicted in FIG. 11.

In another embodiment, the present invention relates to a process for preparation of an amorphous form of the compound of Formula I, the process comprising:
a) providing a solution of a compound of Formula I in one or more solvents;
b) lyophilizing the solution obtained in step a); and
c) optionally isolating the amorphous form of the compound of Formula I.

In another embodiment, the present invention relates to a process for preparation of an amorphous form of the compound of Formula I, the process comprising:
a) milling/grinding a compound of Formula I (e.g., under suitable milling/grinding conditions); and
b) optionally isolating the amorphous form of compound of Formula I.

In another embodiment, the present invention provides a process for the preparation of an amorphous form of compound of Formula I, the process comprising:
a) providing a solution of compound of Formula I in one or more solvents; and
b) removing the solvent (e.g., by evaporation or distillation); and
c) optionally isolating the amorphous form of compound of Formula I.

In another embodiment, the present invention relates to a stable amorphous Form of the compound of Formula I that, after exposure to 40° C./75% RH for a period of about six months, or 25° C./60% RH for a period of at least 12 months, contains less than about 0.5 wt. % total impurities. In one embodiment the stable amorphous Form does not change to any other solid form after exposure to 40° C./75% RH for a period of about six months, or 25° C./60% RH for a period of at least 12 months.

### Premixes of the Compound of Formula I

In another embodiment, the present invention relates to a premix comprising the compound of Formula I and a process for its preparation.

In another embodiment, the present invention relates to a premix comprising the compound of Formula I and one or more pharmaceutically acceptable excipients.

In another embodiment, the present invention relates to a process for the preparation of a premix comprising the compound of Formula I or a pharmaceutically acceptable salt thereof, the process comprising:
a) forming a reaction mixture comprising a compound of Formula I or a pharmaceutically acceptable salt thereof, one or more pharmaceutically acceptable excipients, and one or more solvents;
b) removing the solvent (e.g., by evaporation); and
c) optionally isolating the premix of the compound of Formula I or a pharmaceutically acceptable salt thereof.

In certain embodiments, the one or more pharmaceutically acceptable excipients is selected from, but not limited to polyvinyl pyrrolidone, povidone K-30, povidone K-60, povidone K-90, methylcellulose, methacrylic acid copolymer, hydroxypropylmethyl cellulose, hydroxypropylmethyl cellulose phthalate, hydroxypropylmethyl cellulose acetate succinate (HPMC-AS), hydroxypropylmethyl cellulose, hydroxypropyl cellulose SSL (HPC-SSL), hydroxypropyl cellulose SL (HPC-SL), hydroxypropyl cellulose L (HPC-L), hydroxyethyl cellulose, Soluplus® (polyvinyl caprolactam-polyvinyl acetate-polyethylene glycol graft copolymer (PCL-PVAc-PEG)), gelucire 44/14, ethyl cellulose, D-alpha-tocopheryl polyethylene glycol 1000 succinate, cellulose acetate phthalate, carboxy methyl ethyl cellulose, sodium starch glycolate, any combination of any of the foregoing.

In certain embodiments, removal of a solvent at any stage of the preparation of any crystalline form, amorphous form or premix of the compound of Formula I described herein may include, but is not limited to, centrifugation, crystallization, filtration, extraction, solvent evaporation under atmospheric pressure or reduced pressure/vacuum such as a rotational distillation using Büchi® Rotavapor®, flash evaporation, rotational dying, agitated nutsche filter drying, spray drying, freeze drying, thin film drying, agitated thin film drying, rotary vacuum paddle dryer (RVPD), lyophilization, and the like. In certain embodiments, the solvent may be removed under reduced pressure at a temperature of less than about 100° C., such as less than about 60° C., less than about 40° C., less than about 20° C., less than about 0° C., less than about −20° C., less than about −40° C., less than about −60° C., or less than about −80° C.

In additional embodiments, the compound of Formula I used as the input (starting material) for the preparation of any of the solid forms described herein is a crude mass, a reaction mixture, an amorphous form, one or more crystalline forms or any mixture of any of the foregoing in any proportion.

In additional embodiments, the chemical purity of any of the crystalline forms of the compound of Formula I described herein (as measured by high pressure liquid chromatography) is greater than about 90%, such as about 90.5%, about 91.0%, about 91.5%, about 92.0%, about 92.5%, about 93.0%, about 93.5%, about 94.0%, about 94.5%, about 95.0%, about 95.5%, about 96.0%, about 96.5%, about 97.0%, about 97.5%, about 98.0%, about 98.5%, about 99.0%, about 99.5%, or about 99.9%.

In additional embodiments, the chemical purity of any of the crystalline forms of the compound of Formula I described herein (as measured by high pressure liquid chromatography) is greater than about 90%, such as greater than about 90.5%, greater than about 91.0%, greater than about 91.5%, greater than about 92.0%, greater than about 92.5%, greater than about 93.0%, greater than about 93.5%, greater than about 94.0%, greater than about 94.5%, greater than about 95.0%, greater than about 95.5%, greater than about 96.0%, greater than about 96.5%, greater than about 97.0%, greater than about 97.5%, greater than about 98.0%, greater than about 98.5%, greater than about 99.0%, greater than about 99.5%, or greater than about 99.9%.

In additional embodiments, any of the crystalline forms of the compound of Formula I described herein is about 99.9% chemically pure (as measured by high pressure liquid chromatography).

In further embodiments, any of the crystalline forms of any the compound of Formula I described herein contains less than about 2.0%, such as about 1.9%, about 1.8%, about 1.7%, about 1.6%, about 1.5%, about 1.4%, about 1.3%, about 1.2%, about 1.1%, about 1.0%, about 0.9%, about 0.8%, about 0.7%, about 0.6%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, about 0.01%, or about 0.009% of any one impurity introduced, obtained or produced as a result of the chemical synthesis or degradation, as measured by high pressure liquid chromatography.

In further embodiments, any of the crystalline forms of any the compound of Formula I described herein contains less than about 2.0%, such as less than about 1.9%, less than about 1.8%, less than about 1.7%, less than about 1.6%, less than about 1.5%, less than about 1.4%, less than about 1.3%, less than about 1.2%, less than about 1.1%, less than about 1.0%, less than about 0.9%, less than about 0.8%, less than about 0.7%, less than about 0.6%, less than about 0.5%, less than about 0.4%, less than about 0.3%, less than about 0.2%, less than about 0.1%, less than about 0.09%, less than about 0.08%, less than about 0.07%, less than about 0.06%, less than about 0.05%, less than about 0.04%, less than about 0.03%, less than about 0.02%, less than about 0.01%, or less than about 0.009% of any one impurity (such as any impurity introduced, obtained or produced as a result of the chemical synthesis or degradation of the compound of Formula I), as measured by high pressure liquid chromatography.

In further embodiments, any of the crystalline forms (M-1-M-9) of the compound of Formula I described herein is substantially pure.

In additional embodiments, substantially pure crystalline form of the compound of Formula I means that the crystalline form is substantially free from any other polymorphic form of the compound of Formula I, e.g., the compound of Formula I has polymorphic purity of at least about 90% w/w or more, such as about 95% w/w or more, about 98% w/w or more, or about 99% w/w or more, as characterized by XRPD.

In additional embodiments, any of the crystalline forms of the compound of Formula I described herein comprises one or more other crystalline form(s) of the compound of Formula I or an amorphous form of the compound of Formula I in an amount not more than about 20%, such as not more than about 10%, not more than about 5%, not more than about 2%, not more than about 1%, not more than about 0.5%, not more than about 0.1% or not more than about 0.05%, based on the total volume or weight of the crystalline form. In additional embodiments, any of the crystalline forms of the compound of Formula I described herein comprises one or more other crystalline form(s) or an amorphous form in an amount not detectable (e.g., by XRPD) based the total volume or weight of the crystalline form.

In further embodiments, any of the crystalline forms of the compound of Formula I described herein comprises one or more other crystalline forms of the compound of Formula I in an amount less than about 15%, such as about 10% or less, about 5% or less, or about 2% or less, based on the total volume or weight of the crystalline form.

In further embodiments, any of the crystalline forms of the compound of Formula I described herein comprises one or more other crystalline forms of the compound of Formula I in an amount less than about 5%, such as 1% or less, or about 0.5% or less, based on the total volume or weight of the crystalline form. In further embodiments, any of the crystalline forms of the compound of Formula I described herein comprises one or more other crystalline forms of the compound of Formula I in an amount not detectable based on the total volume or weight of the crystalline form.

In another embodiment, the amorphous form of the compound of Formula I described herein substantially free of any other impurity.

In another embodiment, the amorphous form of the compound of Formula I described herein is substantially pure.

In another embodiment, the amorphous form of the compound of Formula I described herein comprises not more than about 10%, such as not more than about 7%, not more than about 5%, not more than about 3%, not more than about 1% or not more than 0.5% of one or more of the crystalline forms of the compound of Formula I described herein.

In further embodiments, any of the crystalline forms or the amorphous form of the compound of Formula I described herein is characterized by a particle size distribution wherein the doo is about 0.1 μm to about 200 μm.

In further embodiments, any of the crystalline forms or the amorphous form of the compound of Formula I described herein is characterized by a particle size distribution wherein the doo is about 2.0 μm to about 150 μm.

In further embodiments, the one or more solvents used in the preparation of any of the crystalline forms or the amorphous form of the compound of Formula I described herein is selected from the group comprising of ketones, such as methyl ethyl ketone (MEK), methyl isobutyl ketone (MIBK), methyl t-butyl ketone, acetone and the like; alcohol such as ethanol, methanol, n-propanol, 2-propanol (isopropanol), n-butanol, isobutanol, tert-butanol and the like; esters such as ethyl acetate, propyl acetate, n-pentyl acetate, isopropyl acetate, butyl acetate and the like; ethers such as dimethyl ether, diethylether, ethyl methyl ether, 1,4-dioxane, tetrahydrofuran (THF), methyl tetrahydrofuran and like; hydrocarbon such as n-heptane, cyclohexane, n-hexane, benzene, toluene, o-, m- or p-xylene; nitriles such as acetonitrile, propanenitrile; halogenated hydrocarbons such as dichloromethane, dichloroethane, chloroform, tetrachloromethane, chlorobenzene, and like; amides such as dimethyl acetamide (DMA), dimethylformamide (DMF) and like; nitromethane; and water or mixture thereof, preferably the one or more solvent selected from methanol, ethanol, 2-propanol, hexane, heptane, acetonitrile, nitromethane, xylene, acetone, water, and any mixture of any of the foregoing.

In another embodiment, the present invention relates to a pharmaceutical composition comprising a substantially pure solid form of the compound of Formula I according to any of the embodiments described herein and one or more pharmaceutically acceptable excipients.

Certain specific aspects and embodiments of the present application will be explained in greater detail with reference to the following examples, which are provided only for purposes of illustration and should not be construed as limiting the scope of the application in any manner. Variations of the described procedures, as will be apparent to those skilled in the art, are intended to be within the scope of the present application.

### EXAMPLES

### Analytical Methods

Differential scanning calorimetry (DSC) was performed using a Perkin Elmer instrument, Model: DSC 8000. The sample was placed into the respective sample position in the DSC instrument and a blank pan at the reference position. The scan was run in the scanning temperature range of 30° C. to 250° C. (10° C. per minute) using given instrumental parameters and data was collected. A thermogram was recorded by integrating the endothermic peak.

Powder X-ray Diffraction was recorded on an X-Ray powder diffractometer PANalytical X′Pert3; K-Alpha1 [Å](1=1.54060 Å); X'Celerator detector. Prior to analysis, a sufficient quantity of powder sample was ground gently in a mortar and pestle. The ground sample was filled into the groove of the sample holder uniformly and the XRPD graph was recorded within the range of 3 to 40 degrees 2θ.

Thermogravimetric analysis was performed on a Perkin Elmer Pyris 1 TGA. A platinum sample pan was loaded onto the hang down wire and allowed to wait until the balance become stable. The TGA curve was recorded in the temperature range of 30° C. to 400° C. (100° C./minute) with an air flow of 50 mL/minute.

Particle size analysis was performed using a Malvern particle size analyzer. (Mastersizer 3000, Malvern Instrument Ltd).

### Sample Preparation

Weigh accurately 100 mg of sample in 100 ml beaker. Add 3 drops of Tween-80 and 1 mL of water and make a paste with the help of glass rod. Then add 10 ml water and sonicate externally for 10 seconds with continued shaking.

### Instrument Parameters

| Dispersant | Water | |
|---|---|---|
| Dispersant Refractive Index | 1.330 | |
| Level Sensor Threshold | 100 | |
| Particle Type | Non spherical | |
| Scattering Model | Mie | |
| Material Name | Compound of Formula I | |
| Particle Refractive Index | 0.250 | |
| Particle Absorption Index | 0.001 | |
| Particle Density | 0.85 | g/cm³ |
| Background measurement duration | 6 | seconds |
| Sample measurement duration | 6 | seconds |
| Delay between measurements | 0 | seconds |
| Pre-measurement delay | 0 | seconds |

| Obscuration Limit | 10-20% |
|---|---|
| Stirrer RPM | 2000 |
| Analysis Model | General purpose |
| Analysis sensitivity | Normal |
| Fine Mode | Off |

Chemical purity was determined by high pressure liquid chromatography (HPLC).

### Example 1: Preparation of Crystalline Form M-1 of the Compound of Formula I

The compound of Formula I (200 gm) and acetonitrile (1.2 L, 6V) were charged in a round bottom flask (500 mL) at about 25-30° C. The reaction mixture was heated at about 70° C. for 20-25 minutes to completely dissolve the solids. The resulting solution was maintained at room temperature for 40 minutes and allowed to cool gradually. After 1.5 hours, the crystals formed were isolated by filtration, washed with acetonitrile (0.3 L, 1.5 V) and dried in a vacuum tray dryer at about 45-50° C. to yield crystalline Form M-1 of the compound of Formula I. (yield: 70%, 140 gm).

Purity: 99.89%

PSD (d90): 84.9 μm

### Example 2: Preparation of Crystalline Form M-1 of the Compound of Formula I

The compound of Formula I (200 gm) and acetonitrile (1.2 L, 6V) were charged in a round bottom flask (500 mL) at about 20-25° C. The reaction mixture was heated at about 60° C. for 20-25 minutes to completely dissolve the solids. The resulting solution was maintained at room temperature for 30 minutes and allowed to cool gradually. After 1.5 hours, the crystals formed were isolated by filtration, washed with acetonitrile (0.3 L, 1.5 V) and dried in a vacuum tray dryer at about 45-50° C. to yield crystalline Form M-1 of the compound of Formula I. (yield: 70%, 140 gm).

Purity: 99.89%

PSD (d90): 62.4 μm

### Example 3: Preparation of Crystalline Form M-1 of the Compound of Formula I

The compound of Formula I (200 gm) and acetonitrile (1.2 L, 6V) were charged in a round bottom flask (500 mL) at about 25-30° C. The reaction mixture was heated at about 70° C. for 20-25 minutes to completely dissolve the solids. The resulting solution was maintained at room temperature for 40 minutes and then seeded (at about 35-40° C.) with crystalline Form M-1 of compound of Formula I, then allowed to cool gradually. After 1.5 hours crystals formed were filtered, washed with acetonitrile (0.3 L, 1.5 V) and dried in vacuum tray dryer at about 45-50° C. to yield crystalline Form M-1 of compound of Formula I. (yield: 70%, 140 gm).

Purity: 99.89%

PSD (d90): 106 μm

### Example 4: Preparation of Crystalline Form M-2 of Compound of Formula I

Racemic compound of Formula I (200 gm) and methanol (2 L, 10V) were charged in a round bottom flask at about 25-30° C. The reaction mixture was stirred for 1 hour at about 25-30° C. The solid was then isolated by filtration, washed with methanol (0.3 L, 1.5 V) and dried in a vacuum tray dryer at about 45-50° C. The resulting product further purified by chiral chromatography (dichloromethane/methanol 50/50) to obtain chiral pure crystalline Form M-2 of the compound of Formula I. This was lyophilized in acetonitrile (5V), the slurry was washed with MeOH (5 V), then the solid was isolated by filtration and dried in a vacuum tray dryer at about 45-50° C. to yield crystalline Form M-2 of compound of Formula I (yield: 35%, 70 gm).

Purity: 99.8%

PSD (d90): 99.8 μm

### Example 5: Preparation of Crystalline Form M-2 of the Compound of Formula I

Crystalline Form M-1 of the compound of Formula I (200 g) was dried at about 100-120° C. for 2-4 hours to give crystalline Form M-2 of the compound of Formula I (yield: 95%, 190 gm).

### Example 6: Preparation of Crystalline Form M-3 of the Compound of Formula I (Evaporation Method)

The compound of Formula I (300 mg) and ethanol (25 mL) were charged in a round bottom flask (50 mL) at 25-30° C. The reaction mixture was heated to 60-70° C. to dissolve the solid. The solvent was evaporated to obtain crystalline Form M-3 of the compound of Formula I (yield: 90%, 270 mg).

Purity: 99.85%

### Example 7: Preparation of Crystalline Form M-3 of the Compound of Formula I (Slurry Method)

The compound of Formula I (500 mg) and ethanol (5 mL) were charged in a round bottom flask (25 mL) at about 25-30° C. The reaction mass (slurry) was stirred for 10-12 hrs at ambient temperature (RPM 500-600). The slurry was filtered and the resulting solid dried at room temperature to obtain crystalline Form M-3 of the compound of Formula I (yield: 80%, 400 mg).

### Example 8: Preparation of Crystalline Form M-4 of the Compound of Formula I (Slow Evaporation Method)

Crystalline Form M-1 of the compound of Formula I (500 mg) and 40 mL of ethanol-heptane (1:1) were charged in a round bottom flask (100 mL) at about 25-30° C. The reaction mixture was heated then maintained for crystallization at ambient temperature. The solvent was evaporated to obtain crystalline Form M-4 of the compound of Formula I (yield: 90%, 450 mg).

Purity: 99.88%

### Example 9: Preparation of Crystalline Form M-4 of the Compound of Formula I

The compound of Formula I (95 gm) and 570 mL of acetonitrile were charged in a round bottom flask (1 L) at about 25-30° C. The reaction mixture was stirred for 30-45 min. The resulting solid was isolated by filtration, washed with 150 mL of acetonitrile and dried at about 50-70° C. under vacuum in a tray dryer. The dried solid was charged in 475 mL of methanol at about 25-30° C. for 1-2 hrs. The reaction mass was isolated by filtration, washed with methanol and dried at about 50-70° C. under vacuum in a tray dryer to obtain crystalline Form M-4 of the compound of Formula I (yield: 80%, 76 gm).

Purity: 99.9%

PSD (d90): 30-60 μm

### Example 10: Preparation of Crystalline Form M-4 of the Compound of Formula I

Crystalline Form M-3 of the compound of Formula I (200 gm) was dried at about 60° C. for 72 hours to give crystalline Form M-4 of the compound of Formula I (yield: 95%, 190 gm).

### Example 11: Preparation of Crystalline Form M-5 of the Compound of Formula I (Slurry Method)

Crystalline Form M-2 of the compound of Formula I (200 mg) and acetonitrile (3 mL) were charged in a round bottom flask (25 mL) at about 25-30° C. The slurry was stirred overnight at ambient temperature. The slurry was filtered and the resulting solid was dried at room temperature to obtain crystalline Form M-5 of the compound of Formula I (yield: 80%, 160 mg).

Purity: 99.93%

### Example 12: Preparation of Crystalline Form M-6 of the Compound of Formula I (Crash Cooling Method)

Crystalline Form M-2 of the compound of Formula I (200 mg) and nitromethane (3 mL) were charged under liquid nitrogen. The reaction mas solidified at low temperature, then was maintained at room temperature to afford a clear solution. The clear solution further maintained at room temperature for evaporation to obtain crystalline Form M-6 of the compound of Formula I (yield: 40%, 80 mg).

Purity: 99.81%

### Example 13: Preparation of Crystalline Form M-7 of the Compound of Formula I

The compound of Formula I (50 mg) and 2 mL of o-xylene-heptane (1:1) were charged in a round bottom flask (25 mL) at 25-30° C. The reaction mass was heated then maintained at ambient temperature. The solvent was evaporated to obtain crystalline Form M-7 of the compound of Formula I (yield: 80%, 40 mg).

Purity: 99.50%

### Example 14: Preparation of Crystalline Form M-8 of the Compound of Formula I

The compound of Formula I (50 mg) and 4 mL of 2-propanol-hexane (1:1) were charged in a round bottom flask (25 mL) at about 25-30° C. The reaction mass was heated then allowed to crystallize at about 40° C. in a temperature-controlled oven. The solvent was evaporated to obtain crystalline Form M-8 of the compound of Formula I (yield 90%, 45 mg).

Purity: 99.83%

### Example 15: Preparation of Crystalline Form M-9 of the Compound of Formula I (Crash Cooling Method)

Crystalline Form M-2 of the compound of Formula I (200 mg) and methanol (3 mL) were charged under liquid nitrogen. The reaction mass solidified at low temperature, the was maintained at room temperature to afford a clear solution. The clear solution was further maintained at room temperature for evaporation to obtain crystalline Form M-9 of the compound of Formula I (yield: 80%, 160 mg).

Purity: 99.82%

### Example-16: Preparation of Amorphous Form of the Compound of Formula I

The compound of Formula I (0.4 gm) was dissolved in methanol (20 mL) at about 25° C. and the mixture was filtered to make it particle free. The solvent was evaporated using a rotavapor under reduced pressure at about 50° C. The resulting product was re-dissolved in methanol (20 mL) at about 25° C. and the solvent was evaporated using a rotavapor under reduced pressure at about 50° C. for 20 minutes to obtain the amorphous Form of the compound of Formula I (yield 90%, 0.36 gm).

### Example-17: Preparation of Amorphous Form of the Compound of Formula I

A crystalline form of the compound of Formula I (10 gm) was dissolved in water (150 mL) and the resulting mixture was stirred for 15 minutes at room temperature. The solution was then filtered on a hyflo bed, and the resulting solid was washed with water, the water was removed by distillation under vacuum at about 55 to 60° C. to obtain a solid mass. To the solid mass was added heptane (100 mL), which was then removed by distillation. The resulting solid was collected. Heptane (100 mL) was added and the mixture stirred for 2 hours at room temperature. The resulting solid was isolated by filtration, then washed with heptane and dried about 90 to 100° C. for 5 hours to obtain the amorphous Form of the compound of Formula (yield 30%, 3 gm).

### Example-18: Preparation of Amorphous Form of the Compound of Formula I

The compound of Formula I (1.0 gm) was dissolved in acetone (50 mL) at about 25° C. and the resulting mixture stirred at about 60° C. for 4-6 h. The resulting solution was then lyophilized to obtain the amorphous Form of the compound of Formula (yield: 95%, 0.95 gm).

### Example-19: Preparation of Amorphous Form of the Compound of Formula I

The compound of Formula I (1.0 gm) was dissolved in acetonitrile/water (8 ml/2 mL) at about 25° C. and the resulting mixture stirred at about 60° C. The resulting solution was then lyophilized to obtain the amorphous Form of the compound of Formula I (yield: 95%, 0.95 gm).

Purity: 99.8%; PSD (d90): 50-80 μm

### Example-20: Preparation of a Premix of the Compound of Formula I with Hydroxypropyl Methylcellulose (HPMC)

The compound of Formula I (200 mg) and hydroxypropyl methylcellulose (100 mg) were dissolved in dichloromethane (20 mL) at 40° C. The resulting solution was filtered to make it particle-free. The solvent was then evaporated under vacuum with stirring at 175 rpm at 50° C. The resulting solid was dried at 50° C. for 15 minutes to obtain the premix of the compound of Formula I. (yield: 95%, 385 mg).

All references and patent publications cited herein are hereby incorporated by reference.

1. A solid form of a compound of Formula I: 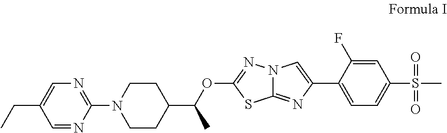 [SMILES: CCc1cnc(N2CCC([C@H](C)Oc3nn4cc(-c5ccc(S(C)(=O)=O)cc5F)nc4s3)CC2)nc1]
wherein the solid form is selected from:
A. crystalline Form M-1 characterized by:
i) an X ray powder diffraction spectrum (XRPD) exhibiting characteristic peaks at 6.0±0.2, 8.1±0.2 and 17.3±0.2° 2θ;
ii) an X ray powder diffraction spectrum (XRPD) substantially as depicted in FIG. 1;
iii) a differential scanning calorimetry (DSC) plot exhibiting endothermic peaks at about 111° C. and about 141.7° C.; or
iv) any combination of any of the foregoing;
B. crystalline Form M-2 characterized by:
i) an X ray powder diffraction spectrum (XRPD) exhibiting characteristic peaks at 6.9±0.2, 10.6±0.2, 16.6±0.2, 19.2±0.2 and 20.4±0.2° 2θ;
ii) an X ray powder diffraction spectrum (XRPD) substantially as depicted in FIG. 2;
iii) a differential scanning calorimetry (DSC) plot exhibiting an endothermic peak at about 144° C.; or
iv) any combination of any of the foregoing;
C. crystalline Form M-3 characterized by:
i) an X ray powder diffraction spectrum (XRPD) exhibiting characteristic peaks at 7.1±0.2, 13.3±0.2, 17.6±0.2 and 21.8±0.2° 2θ;
ii) an X ray powder diffraction spectrum (XRPD) substantially as depicted in FIG. 3;
iii) a differential scanning calorimetry (DSC) plot exhibiting an endothermic peak at about 113° C.; or
iv) any combination of any of the foregoing;
D. crystalline Form M-4 characterized by:
i) an X ray powder diffraction spectrum (XRPD) exhibiting characteristic peaks at 8.8±0.2, 11.3±0.2, 17.8±0.2, 19.0±0.2, 23.1±0.2 and 25.8±0.2° 2θ;
ii) an X ray powder diffraction spectrum (XRPD) substantially as depicted in FIG. 4;
iii) a differential scanning calorimetry (DSC) plot substantially as depicted in FIG. 5;
iv) a Differential Scanning calorimetry (DSC) plot exhibiting an endothermic peak at about 107° C.; or
v) any combination of any of the foregoing;
E. crystalline Form M-5 characterized by:
i) an X ray powder diffraction spectrum (XRPD) exhibiting characteristic peaks at 4.3±0.2, 7.8±0.2, 10.2±0.2, 16.3±0.2 and 16.8±0.2° 2θ;
ii) an X ray powder diffraction spectrum (XRPD) substantially as depicted in FIG. 6;
iii) a differential scanning calorimetry (DSC) plot exhibiting an endothermic peak at about 148° C.; or
iv) any combination of any of the foregoing;
F. crystalline Form M-6 characterized by:
i) an X ray powder diffraction spectrum (XRPD) exhibiting characteristic peaks at 5.6±0.2, 9.7±0.2 and 19.8±0.2° 2θ;
ii) an X ray powder diffraction spectrum (XRPD) substantially as depicted in FIG. 7;
iii) a differential scanning calorimetry (DSC) plot exhibiting endothermic peaks at about 87° C., about 112° C. and about 142° C.; or
iv) any combination of any of the foregoing;
G. crystalline Form M-7 characterized by:
i) an X ray powder diffraction spectrum (XRPD) exhibiting characteristic peaks at 4.3±0.2, 8.7±0.2, 10.6±0.2, 16.4±0.2 and 18.5±0.2° 2θ;
ii) an X ray powder diffraction spectrum (XRPD) substantially as depicted in FIG. 8;
iii) a differential scanning calorimetry (DSC) plot comprising endothermic peaks at about 100° C.; or
iv) any combination of any of the foregoing;
H. crystalline Form M-8 characterized by:
i) an X ray powder diffraction spectrum (XRPD) exhibiting characteristic peaks at 4.6±0.2, 9.3±0.2, 12.3±0.2, 15.5±0.2 and 17.0±0.2° 2θ;
ii) an X ray powder diffraction spectrum (XRPD) substantially as depicted in FIG. 9;
iii) a differential scanning calorimetry (DSC) plot exhibiting an onset at about 127° C. and an endothermic peak at about 133° C.; or
iv) any combination of any of the foregoing;
I. crystalline Form M-9 characterized by:
i) an X ray powder diffraction spectrum (XRPD) exhibiting characteristic peaks at 11.5±0.2, 13.3±0.2, 15.6±0.2, 17.9±0.2, 19.0±0.2, 23.2±0.2 and 25.8±0.2° 2θ;
ii) an X ray powder diffraction spectrum (XRPD) substantially as depicted in FIG. 10;
iii) a differential scanning calorimetry (DSC) plot exhibiting an onset at about 108° C. and an endothermic peak at about 142° C.; or
iv) any combination of any of the foregoing;
J. an amorphous form characterized by an X ray powder diffraction spectrum (XRPD) substantially as depicted in FIG. 11.

2. The solid form of as claimed in claim 1, wherein
a) Form M-1 further comprises characteristic XRPD peaks at 4.5±0.2, 7.0±0.2, 10.5±0.2, 12.5±0.2, 14.9±0.2, 15.1±0.2, 15.4±0.2, 16.6±0.2, 18.6±0.2, 19.3±0.2, 20.4±0.2, 25.8±0.2 and 26.4±0.2° 2θ;
b) Form M-2 further comprises characteristic XRPD peaks at 4.5±0.2, 12.5±0.2, 14.1±0.2, 14.9±0.2, 15.4±0.2, 17.9±0.2 and 20.8±0.2° 2θ;
c) Form M-3 further comprises characteristic XRPD peaks at 4.3±0.2, 11.1±0.2, 15.2±0.2, 15.7±0.2, 16.0±0.2, 17.5±0.2, 18.8±0.2, 20.2±0.2, 21.7±0.2, 23.2±0.2 and 25.7±0.2° 2θ;
d) Form M-4 further comprises characteristic XRPD peaks at 11.5±0.2, 12.3±0.2, 19.4±0.2, 22.0±0.2, 23.1±0.2, 24.5±0.2 and 25.9±0.2° 2θ;
e) Form M-5 further comprises characteristic XRPD peaks at 7.6±0.2, 12.3±0.2, 17.8±0.2, 19.4±0.2 and 20.6±0.2° 2θ; and
f) Form M-6 further comprises characteristic XRPD peaks at 15.9±0.2, 17.3±0.2, 18.6±0.2 and 20.3±0.2°.

3. A process for the preparation of crystalline Form M-1 of the compound of Formula I as claimed in claim 1, the process comprising:
a) dissolving a compound of Formula I in one or more solvents;
b) heating the reaction mixture;
c) cooling the heated mixture obtain in step b);
d) optionally seeding the reaction mixture with crystalline Form M-1 of the compound of Formula I; and
e) optionally isolating the crystalline Form M-1 of the compound of Formula I.

4. A process for the preparation of crystalline Form M-2 of the compound of Formula I as claimed in claim 1, the process comprising:
a) mixing racemic compound of Formula I in one or more solvents;
b) stirring the reaction mixture;
c) isolating the resulting solid;
d) obtaining the desired isomer of the crystalline Form M-2 of the compound of Formula I; and
e) optionally isolating the crystalline Form M-2 of the compound of Formula I.

5. A process for the preparation of crystalline Form M-2 of the compound of Formula I as claimed in claim 1, the process comprising heating a compound of Formula I to give the crystalline Form M-2 of the compound of Formula I.

6. A process for the preparation of crystalline Form M-3 of the compound of Formula I as claimed in claim 1, the process comprising:
a) dissolving a compound of Formula I in one or more solvents;
b) heating the reaction mixture; and
c) optionally isolating the crystalline Form M-3 of the compound of Formula I.

7. A process for the preparation of crystalline Form M-4 of the compound of Formula I as claimed in claim 1, the comprising:
a) dissolving a compound of Formula I in a mixture of solvents;
b) heating the reaction mixture;
c) crystallizing the reaction mixture; and
d) optionally isolating the crystalline Form M-4 of the compound of Formula I.

8. A process for the preparation of crystalline Form M-4 of compound of Formula I as claimed in claim 1, the process comprising:
a) dissolving a compound of Formula I in one or more solvents;
b) stirring the reaction mixture; and
c) optionally isolating the crystalline Form M-4 of the compound of Formula I.

9. A process for the preparation of crystalline Form M-4 of the compound of Formula I as claimed in claim 1, the process comprising heating crystalline Form M-3 of the compound of Formula I to give the crystalline Form M-4 of the compound of Formula I.

10. A process for the preparation of crystalline Form M-5 of the compound of Formula I as claimed in claim 1, the process comprising:
a) dissolving a compound of Formula I in one or more solvents;
b) stirring the reaction mass at ambient temperature; and
c) optionally isolating the crystalline Form M-5 of the compound of Formula I.

11. A process for the preparation of crystalline Form M-6 of the compound of Formula I as claimed in claim 1, the process comprising:
a) dissolving a compound of Formula I in one or more solvents under liquid nitrogen conditions to afford a solid mass;
b) maintaining the solid mass at room temperature to afford a clear solution; and
c) optionally isolating the crystalline Form M-6 of the compound of Formula I.

12. A process for the preparation of crystalline Form M-7 of the compound of Formula I as claimed in claim 1, the process comprising:
a) dissolving a compound of Formula I in one or more solvents;
b) heating the reaction mixture obtained in step a);
c) evaporating the solvent to obtain crystalline Form M-7; and
d) optionally isolating the crystalline Form M-7 of the compound of Formula I.

13. A process for the preparation of crystalline Form M-8 of the compound of Formula I as claimed in claim 1, the process comprising:
a) dissolving a compound of Formula I in a mixture of solvents;
b) heating the reaction mixture of step a);
c) cooling the reaction mixture obtained from step b); and
d) optionally isolating the crystalline Form M-8 of the compound of Formula I.

14. A process for the preparation of crystalline Form M-9 of the compound of Formula I as claimed in claim 1, the process comprising:
a) dissolving compound of Formula I in one or more solvents under liquid nitrogen conditions to afford a solid mass;
b) maintaining the solid mass at room temperature to afford a clear solution; and
c) isolating the crystalline Form M-9 of the compound of Formula I.

15. A process for the preparation of an amorphous form of the compound of Formula I as claimed in claim 1, the process comprising:
a) providing a solution of a compound of Formula I in one or more solvents;
b) lyophilizing the solution obtained in step a); and
c) optionally isolating the amorphous form of the compound of Formula I.

16. A process for the preparation of an amorphous form of the compound of Formula I as claimed in claim 1, the process comprising:
a) providing a solution of a compound of Formula I in one or more solvents;
b) removing the solvent; and
c) optionally isolating the amorphous form of the compound of Formula I.

17. The process according to claim 3, wherein the compound of Formula I provided in step a) is selected from an amorphous form, a crystalline form, or any mixture thereof.

18. A process for the preparation of a premix of a compound of Formula I the process comprising:
a) mixing one or more pharmaceutically acceptable excipients, a compound of Formula I as claimed in claim 1, and one or more solvents;
b) removing the solvent; and
c) optionally isolating the premix of the compound of Formula I.

19. A pharmaceutical composition comprising a solid form of a compound of Formula I as claimed in claim 1 and a pharmaceutically acceptable excipient.

20. A solid form of a compound of Formula I as claimed in claim 1, wherein the solid form is substantially free of other solid forms of the compound of Formula I.
